SEQUENCE LISTING (23 sequences)

SEQ ID NO: 1 - DNA, 10 bp, Artificial Sequence
  Description of Artificial Sequence Synthetic PNA oligonucleotide which does not occur in nature
tcnnggtaca

SEQ ID NO: 2 - DNA, 10 bp, Artificial Sequence
  Description of Artificial Sequence Synthetic PNA oligonucleotide which does not occur in nature
catngnatcg

SEQ ID NO: 3 - DNA, 10 bp, Artificial Sequence
  Description of Artificial Sequence Synthetic PNA oligonucleotide which does not occur in nature
ugunnnttgn

SEQ ID NO: 4 - DNA, 10 bp, Artificial Sequence
  Description of Artificial Sequence Synthetic PNA oligonucleotide which does not occur in nature
ngnutntnug

SEQ ID NO: 5 - DNA, 12 bp, Artificial Sequence
  Description of Artificial Sequence Synthetic PNA oligonucleotide which does not occur in nature
cuggnntungnc

SEQ ID NO: 6 - DNA, 12 bp, Artificial Sequence
  Description of Artificial Sequence Synthetic PNA oligonucleotide which does not occur in nature
gtntaattnnag

SEQ ID NO: 7 - DNA, 12 bp, Artificial Sequence
  Description of Artificial Sequence Synthetic PNA oligonucleotide which does not occur in nature
ngtcgnnggucu

SEQ ID NO: 8 - DNA, 12 bp, Artificial Sequence
  Description of Artificial Sequence Synthetic PNA oligonucleotide which does not occur in nature
agacnttngant

SEQ ID NO: 9 - DNA, 10 bp, Artificial Sequence
  Description of Artificial Sequence Synthetic PNA oligonucleotide which does not occur in nature
tcnnnntaca

SEQ ID NO: 10 - DNA, 12 bp, Artificial Sequence
  Description of Artificial Sequence Synthetic PNA oligonucleotide which does not occur in nature
ngtntcgtnccg

SEQ ID NO: 11 - DNA, 18 bp, Artificial Sequence
  Description of Artificial Sequence Synthetic PNA oligonucleotide which does not occur in nature
aacgggataactgcacct

SEQ ID NO: 12 - DNA, 10 bp, Artificial Sequence
  Description of Artificial Sequence Synthetic PNA oligonucleotide which does not occur in nature
tcaaggtaca

SEQ ID NO: 13 - DNA, 10 bp, Artificial Sequence
  Description of Artificial Sequence Synthetic PNA oligonucleotide which does not occur in nature
tgtaccttga

SEQ ID NO: 14 - DNA, 12 bp, Artificial Sequence
  Description of Artificial Sequence Synthetic PNA oligonucleotide which does not occur in nature
agacnttngnnt

SEQ ID NO: 15 - DNA, 12 bp, Artificial Sequence
  Description of Artificial Sequence Synthetic PNA oligonucleotide which does not occur in nature
gtntanttnnag

SEQ ID NO: 16 - DNA, 10 bp, Artificial Sequence
  Description of Artificial Sequence Synthetic PNA oligonucleotide which does not occur in nature
ttgannttag

SEQ ID NO: 17 - DNA, 10 bp, Artificial Sequence
  Description of Artificial Sequence Synthetic PNA oligonucleotide which does not occur in nature
tgtannttga

SEQ ID NO: 18 - DNA, 12 bp, Artificial Sequence
  Description of Artificial Sequence Synthetic PNA oligonucleotide which does not occur in nature
ngtcgnnggucu

SEQ ID NO: 19 - DNA, 12 bp, Artificial Sequence
  Description of Artificial Sequence Synthetic PNA oligonucleotide which does not occur in nature
ucggnntungnc

SEQ ID NO: 20 - DNA, 10 bp, Artificial Sequence
  Description of Artificial Sequence Synthetic PNA oligonucleotide which does not occur in nature
ctaaggtcaa

SEQ ID NO: 21 - DNA, 10 bp, Artificial Sequence
  Description of Artificial Sequence Synthetic PNA oligonucleotide which does not occur in nature
tcaaggtaca

SEQ ID NO: 22 - DNA, 10 bp, Artificial Sequence
  Description of Artificial Sequence Synthetic PNA oligonucleotide which does not occur in nature
ctaaggtcaa

SEQ ID NO: 23 - DNA, 10 bp, Artificial Sequence
  Description of Artificial Sequence Synthetic PNA oligonucleotide which does not occur in nature
tcnnggtaca

(19) United States
(12) Patent Application Publication
(10) Pub. No.: US 2010/0151460 A1
(43) Pub. Date: Jun. 17, 2010

(54) Systems and Methods for Biological and Chemical Detection, Comprising Automatic Selection of Reagent Sets

(76) Inventor: Lars Winther, Smoerum (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/516,678

(22) PCT Filed: Nov. 29, 2007
(86) PCT No.: PCT/IB07/04708

§ 371 Date: May 28, 2009

Related U.S. Application Data
(60) Provisional application No. 60/861,956, filed on Dec. 1, 2006.

(51) Int. Cl. C12Q 1/68; G01N 33/53; C12M 1/34; G06F 19/00

(52) U.S. Cl. 435/6; 435/7.9; 435/287.2; 435/287.3; 702/19

(57) ABSTRACT

The instant invention relates to systems of reagents and methods for the detection of chemical and biological targets in a sample. Some embodiments comprise methods for automatically selecting a set of reagents to detect one or more targets in a sample, wherein the set of reagents comprises at least two layers for detection of a first target, and at least two layers for detection of a second target, wherein the set comprises reagents that are at least partially redundant. In some embodiments, the redundancy is created by at least one degenerate reagent such that the reagent may interact specifically with more than one other component of a detection system or sample. In some embodiments, the system or method also includes reagent containers with a computer-generated code which may further serve to match targets to appropriate reagents.

### RELATED APPLICATIONS

This invention claims priority to U.S. Provisional Patent Application No. 60/861,956, filed Dec. 1, 2006, which is incorporated herein by reference.

### FIELD OF THE INVENTION

The instant invention relates to systems of reagents and methods for the detection of chemical and biological targets in a sample.

Some embodiments comprise methods for automatically selecting a set of reagents to detect one or more targets in a sample, wherein the set of reagents comprises at least two layers for detection of a first target, and at least two layers for detection of an optional second target, wherein the set optionally comprises reagents that are at least partially redundant. In some embodiments, the redundancy is created by at least one degenerate reagent such that the reagent may interact specifically with more than one other component of a detection system or sample. In some embodiments, the system or method also includes reagent containers with a computer-generated code which may further serve to match targets to appropriate reagents.

### BACKGROUND AND SUMMARY OF THE INVENTION

Diagnostic or detection assays commonly used in biology and chemistry, such as Western, Northern, or Southern blots, immunohistochemistry (IHC), immunocytochemistry, in situ hybridization (ISH), ELISA, and the like, all operate on the basic principle that a target in a sample is detected by contacting the target with a probe which it specifically recognizes, which leads to a detectable change in the sample that registers as a signal. For instance, the probe may be linked, either directly or indirectly, to a detectable label, such as a fluorophore, chromophore, or an enzymatic or radioactive tag, which provides the signal. Polymeric detection reagents and conjugates are also compatible with this invention.

Some detection systems also provide ways of enhancing the signal from the target. For example, the label's signal may be enhanced by increasing the number of detectable labels used to detect each target, or by instrumentation that may amplify the signal. If the target is an antigen, a multiple-antibody system may amplify the detection signal. For instance, the target may first be bound by a primary antibody probe, which, in turn, is capable of binding many secondary antibodies, which, in turn, may optionally be recognized by tertiary antibodies, which act as amplification layers. The detectable label, then, may be present on or recognized by the outermost amplification layer. This method, thus, increases the strength of the signal from the detection of each target, as many detectable labels become associated with each target rather than only one or a few.

In some cases, one may also wish to detect more than one target in a sample, either in separate procedures, or simultaneously on the same portion of the sample. For example, an experimenter may wish to test a biological sample for the presence of several different genetic targets or protein targets in order to assist in a diagnostic procedure. It may be more efficient, in some settings, to perform those assays simultaneously or immediately following one another on the same part of the sample, so that the number of steps involved is minimized.

The present invention allows for a closed reagent system for detecting one target or more than one target in a sample. The set of reagents is automatically selected, which may help to ensure accuracy. For example, the system may choose a correct set of reagents in pre-optimized amounts and in the correct order, thus reducing human errors in carrying out the protocols. In some embodiments, the system may also choose the reaction conditions, and be programmed with information concerning cross-reactivities of various reagents. Accordingly, the reagent sets, systems, and methods of the present invention may lessen the risk of errors in diagnosis due to false positives or false negatives in a diagnostic assay, by allowing for greater uniformity of application.

The instant invention includes a system and method for automatically selecting a set of reagents for a detection protocol, which set may be used to detect one or more than one target in a sample. The selected set of reagents comprises at least two layers of reagents for each target to be detected, and includes at least one redundant reagent. The redundant reagent, for example, can be replaced by at least one other detection reagent in the set. Hence, a detection assay, run with the selected reagent set, can be optimized to choose the appropriate reagent from among the redundant reagents in the set. In some embodiments, the set of reagents also includes at least one degenerate reagent, interacts with more than one other reagent in the set, making that reagent redundant. In some embodiments, the degenerate reagent, because of its flexibility of interactions, could be used in the detection of more than one target.

Such redundancies and reagent interchangeabilities may increase the efficiency of some detection assays, as they allow for mixing and matching between different reagents. When degenerate reagents are also used, that interchangeability may allow for fewer detection reagents in a particular system, so that multi-target detection is simpler to carry out and is associated with less risk of unwanted interactions between detection reagents. Redundancies may also allow a researcher to choose from more than one type detectable label. That mixing and matching may also expand the uses of a particular set of detection reagents, so that a system can be more easily adapted to detecting several different targets, depending upon the user's needs. For instance, if a particular detectable label recognizes more than one probe, then that label could be used in more than one detection assay, thus reducing the number of labels needed for a given set of assays.

In some systems of the invention, the degenerate reagent contains at least one degenerate molecular code such that the same site of the reagent is capable of specifically binding to more than one other molecule. In some embodiments, degenerate nucleic acid hybridization may be used to create a degenerate molecular code. In other embodiments, an epitope of an antigen may specifically recognize more than one antibody, for example.

Alternatively, degeneracy in a reagent could be created with reagents that contain more than one binding site, each for a different binding partner. For example, if nucleic acid hybridization is used for the detection reagents to interact, a degenerate reagent may contain two different recognition sequences. If antigen-antibody interactions are used, one may design an antigen such that it has more than one different epitope. Yet further, molecular entities can be constructed using chemical linkers and polymers such that they bridge two different binding elements together in one molecule.

To aid in automated selection of the reagent set, simple algorithms may be used to create a set of information about each reagent, such as its function in the detection method, which targets it is used to detect, what other detection reagents it interacts with, any redundancies or degeneracies it has, and at what stage and concentration it is applied to the sample. In some embodiments, the automated selection of the reagent set occurs fully or in part through a computer-generated code on the reagent containers. The computer-generated code may be used to retain the above information so that when a user desires to detect a particular target, the correct set of reagents is selected and organized, and redundant reagents are noted. Examples include bar codes and sku codes, but other known software-readable signals would suffice. Such automated reagent selection is compatible with various known automated or semi-automated detection apparatuses. Further, in some cases, the automated reagent selection may also allow for certain parts of the detection procedure on the sample to be carried out manually.

The instant methods and resulting reagent systems are compatible with a large variety of samples and are adaptable to a large number of targets, probes, and detectable labels. For instance, the present invention is useful in immunohistochemistry applications (IHC) and in situ hybridization (ISH), and can be applied to other detection methods as well. Other detection that may be compatible with this invention include, for example, immunocytochemistry (ICC), flow cytometry, enzyme immuno-assays (EIA), enzyme linked immuno-assays (ELISA), blotting methods (e.g. Western, Southern, and Northern), labeling inside electrophoresis systems or on surfaces or arrays, and precipitation, among others.

Such detection formats, for example, are useful in research as well as in diagnosing diseases or conditions. Further, if multiple targets are detected, such systems may be useful in analyzing expression patterns of genes or levels of proteins within a sample.

### BRIEF DESCRIPTION OF THE DRAWINGS

FIG. 1 illustrates an exemplary two or three detection reagent system which is compatible with the invention. The probe in this example is connected to a detectable label by hybridization of nucleic acid analog segments, one or more of which may comprise a degenerate molecular code. Both the probe and label are each present on larger molecular entities. An adaptor unit, shown in the middle panel is optional, but may, in some cases, serve to link the probe and detectable label through an intermediate set of nucleic acid hybridizations, and may also serve as an amplification layer. The nucleic acid analog segments may be present on one or more of the molecular entities in the system.

FIG. 2 illustrates an exemplary system and method compatible with the invention in which a target antigen bound to a primary antibody is recognized by a recognition unit comprising a secondary antibody probe. The recognition unit is specifically hybridized to a detection unit via the nucleic acid analog segments on each unit. In such a system, either the detection unit or the recognition unit molecular entities may comprise a degenerate molecular code. The system shown in this figure has 3 layers of detection reagents above the target: the primary antibody, recognition unit with probe, and detection unit with detectable label.

FIG. 3 illustrates an exemplary three or higher-layer detection system and method compatible with the invention wherein a target antigen bound to a primary antibody is recognized by a recognition unit comprising a secondary antibody probe and a nucleic acid analog segment. The recognition unit specifically hybridizes to an adaptor unit comprising nucleic acid analog segments that specifically hybridize to the recognition unit and a detection unit. Hence, there are four layers in this system above the target: the primary antibody, secondary antibody probe molecular entity, adaptor, and detection unit with detectable label. Any of the detection reagents may comprise degenerate molecular codes in their nucleic acid analog segments, for example, or may be replaceable with a redundant reagent.

FIGS. 4-6 illustrate exemplary non-natural bases and base-pairings which may be used in the instant invention to produce nucleic acid-based degenerate molecular codes in detection reagents.

FIG. 7 illustrates an exemplary system in which an antigen is degenerate and is recognized by more than one specific antibody. For instance, the antigen may incorporate more than one epitope, or an epitope that is recognized by more than one different antibody. Alternatively, the same epitope could be bound specifically by different antibodies. In this example, each different antibody carries a different detectable label, leading to redundancy, as either one of the antibodies could be selected for a detection experiment. That redundancy allows for a choice among detection labels.

FIG. 8 presents two illustrations showing, first, how a set of reagents may be include one redundant reagent. In the top illustration, the same target may be detected by either A, B, and C, or by A, X, and C. X and B are redundant as each can take the place of the other. The second panel illustrates how two targets may be detected by overlapping sets of reagents as one reagent is degenerate. Target 1 is detected by A, B, and C, while Target 2 is detected by P, B, and D. Reagent B is able to interact with all of P, A, C, and D, due to a degeneracy in its recognition properties. Thus, reagent B is degenerate. Reagents C and D are redundant in that B could interact with either of them. If reagents C and D are detectable labels, that redundancy allows the experimenter detecting the targets to select the more appropriate label for the experiment.

FIG. 9 illustrates an example of redundancy of detection reagents in the systems and methods according to some embodiments of the invention. In FIG. 9, the molecular entity carrying the detectable label may be used in the detection of Target 1 by binding directly to the molecular entity carrying the probe. In the detection of Targets 2 and 3, that same entity binds to an adaptor unit. Thus, the molecular entity is redundant in that it can be used in more than one detection assay in a system. The adaptor unit shown in FIG. 8 is also redundant in that it can be used in the detection of both Targets 2 and 3.

### DETAILED DESCRIPTION OF THE INVENTION

### Definitions

Redundant or redundancy, as applied to the set of reagents herein, applies to a molecule or reagent that can be replaced with another reagent in the same set.

Degenerate, as used herein, applies to a molecule that is able to specifically bind to more than one other molecule in a set of reagents. A degenerate molecular code, herein, is one way to produce degeneracy in a detection reagent. The term applies to a code at the molecular structure level that recognizes more than one other molecular structure.

A computer-generated code, as used herein, includes any code that may be created or interpreted by computer hardware and/or software, including but not limited to numerical, color, and letter codes.

Automated or automatic, and the like, refer to non-manual methods.

Set of as used herein means two or more of any item.

Detection reagent as used herein means a reagent that is used to detect a target in a sample by either directly recognizing the target or by directly recognizing another detection reagent that, in turn, directly recognizes the target.

Sample, as used herein, refers to any composition potentially containing a target.

Target, as used herein, refers to any substance present in a sample that is capable of detection.

The term recognize, and similar terms, when applied to a target or detection reagent herein, means to render the target detectable by a detectable label. Recognition includes, for example, reacting with a target, directly binding to a target, and indirectly reacting with or binding to a target.

The terms bind, binding, and similar terms, when applied to the instant targets and detection reagents, mean an event in which one substance physically interacts with another. Specific, specific for, or specifically and similar terms are used to indicate that the binding between two or more molecular entities is through specific interactions rather than through non-specific aggregation, for example. Specific hybridization and like terms as used herein refer to the specific binding of two single-stranded nucleic acid segments to create double-stranded nucleic acids.

Amplify, amplification, and similar terms, mean an increase in the observed intensity of a signal from a detectable label.

A protein herein is used in the broadest possible sense, and includes any molecule comprising a sequence of amino acids, such as a short peptide, peptide hormone, or protein fragment, and larger molecules including antibodies, enzymes, glycoproteins, lipoproteins, etc.

Antibody, as used herein, means an immunoglobulin or a fragment thereof, and encompasses any polypeptide comprising an antigen-binding site regardless of the source, method of production, and other characteristics.

An antigen, as used herein, refers to any substance recognized by an antibody.

As used herein, a nucleic acid, nucleic acid sequence, or nucleic acid segment is defined in the broadest possible sense and includes a variety of natural nucleic acids as well as nucleic acid analogs. For instance, nucleic acid may be any nucleobase sequence comprising any oligomer, polymer, or polymer segment, wherein an oligomer means a sequence of two or more backbone monomer units. Backbones include any substance capable of forming an oligomer, including DNA, RNA, PNA, LNA, and any modified or substituted backbone. Nucleobases (or bases) may be, for example, natural bases such as adenine (A), cytosine (C), guanine (G), thymine (T), and uracil (U), as well as any non-natural base.

As used herein, the terms base and nucleobase refer to any purine-like or pyrimidine-like molecule that may be comprised in a nucleic acid segment or nucleic acid analog segment.

A non-natural base, as used herein, means any nucleobase other than Adenine, A; Guanine, G; Uracil, U; Thymine, T; or Cytosine, C. Non-natural bases also include molecules elsewhere termed “base analogs.”

A non-natural backbone unit includes any type of backbone unit to which a nucleobase may be attached that is not a ribose-phosphate (RNA) or a deoxyribose-phosphate (DNA) backbone unit.

As used herein, the terms nucleic acid analog segment, nucleic acid analog, or nucleic acid analog sequence mean any oligomer, polymer, or polymer segment, comprising at least one monomer that comprises a non-natural base and/or a non-natural backbone unit.

As used herein, all numbers are approximate, and may be varied to account for errors in measurement and rounding of significant digits.

### Detection Reagents

Example detection reagents compatible with this invention include reagents such as probes, which specifically bind to a target in a sample, or detectable labels, which create a signal which can be detected, to indicate the presence and/or concentration of a target, and various reagents that link probes and detectable labels together, such as molecular adaptors, which physically connect the probe and detectable label. Adaptors may in some cases serve to amplify a detection signal, for instance if many adaptor molecules bind to each probe. An example of such an amplifying adaptor is a secondary antibody, which recognizes the constant region of a primary antibody probe, such that many secondary antibodies bind to each primary antibody in the sample. If a detectable label recognizes the secondary antibody, for instance, an enzyme with a colored substrate, then many such labels will become associated with each probe-target in the sample.

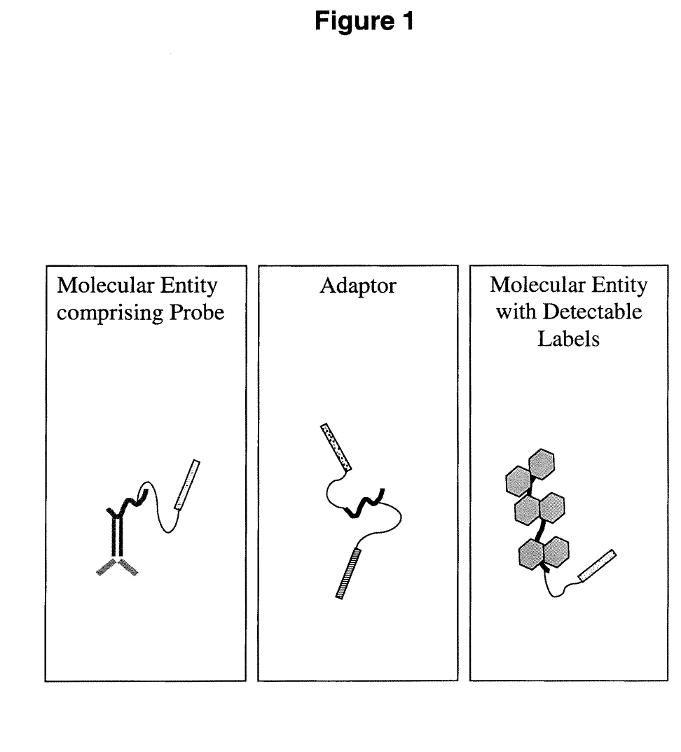

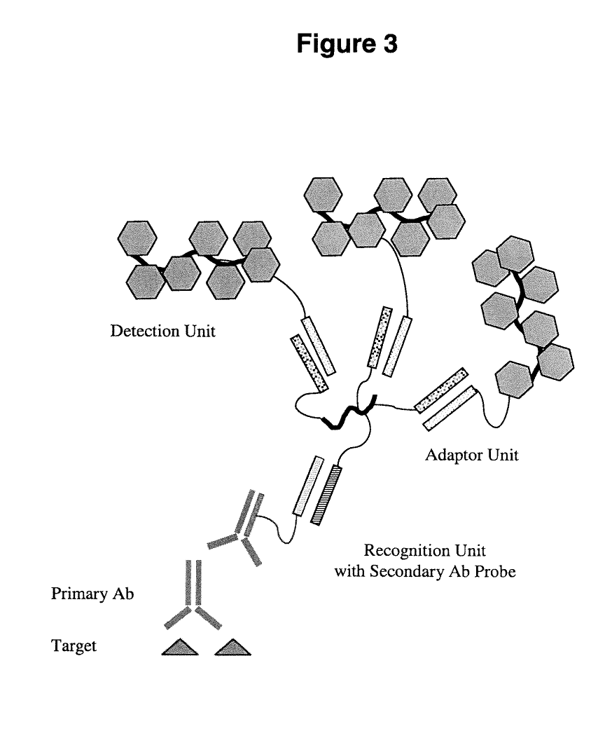

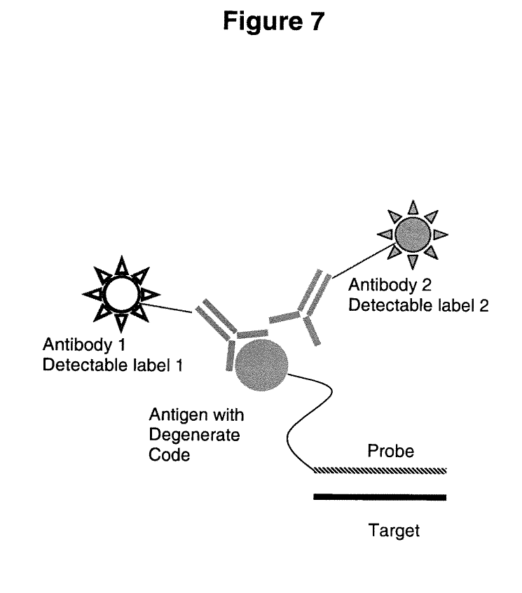

Any or all of the detection reagents described herein may be present in isolation, or may form part of larger molecular entities, for instance. One example system of larger, interacting molecular entities is shown in FIGS. 1-3, in which a probe and detectable label are each present on an entities that interact through nucleic acid hybridization. The nucleic acid segments of the molecular entities may be attached to the probe and detectable label, for instance, via chemical linkers and polymers. There may also be multiple nucleic acid segments, or more than one nucleic acid segment on at least one of the molecular entities, such that the entity is able to interact with more than one specific binding partner, depending upon the experimental protocol. Alternatively, FIG. 7 illustrates how antigen-antibody interactions can physically connect different probe-label combinations together.

### Automated Reagent Selection and Redundant Reagents

The instant invention allows for the automatic selection of a set of reagents to detect one or more targets in a sample, such as for diagnostic purposes. In such methods, an algorithm may be used to organize the detection reagents into layers (i.e. probes, adaptors or amplifiers, and detectable labels) of at least 2 or at least 3. The algorithm may also specify the targets the reagents are compatible with. The algorithm may further specify the other detection reagents that each reagent selected interacts with, including intended interactions to produce a signal, and any unwanted interactions that might interfere with labeling. Those methods may, in some embodiments, select a set of reagents in which the set includes at least two layers of reagents to detect a first target, and at least two layers of reagents to detect an optional second target, wherein at least one reagent in the set is redundant. The redundant reagent may used to replace another reagent in the set, if needed, or it may not be used in the detection protocol.

In some embodiments, at least one of the reagents in the set is a degenerate reagent, and thus, is able to interact with more than one other reagent in the set. Degenerate reagents may allow other reagents in the set to be redundant, as the degenerate reagents can interact with more than one other reagent. FIGS. 7-9, described above, provide illustrations of redundancies and degeneracies according to the invention.

The automated method may be put into action by a computer and associated software, for example, so that when a user selects a particular target to identify, the detection system is able to identify an appropriate set of reagents. When a user selects more than one target, in some embodiments, the system would be able to select complementary reagents, including redundant reagents.

For example, if using a computer and software to select the reagents, each reagent could be coded based upon a specific set of parameters, such as, first, its level in the detection method. A probe, for example, may be at level 1, as it is intended to interact with a target. Nevertheless, if a probe indirectly interacts with a target through another entity, or if a blocking step is employed first in the reaction, a probe could be assigned to a higher level in the organization, with the blocker or other reagent taking the first level. An adaptor, if used, could be at level 2, if it directly interacts with the probe, and a detectable label may be at level 2 or 3 or higher, depending upon whether an adaptor is used, for example.

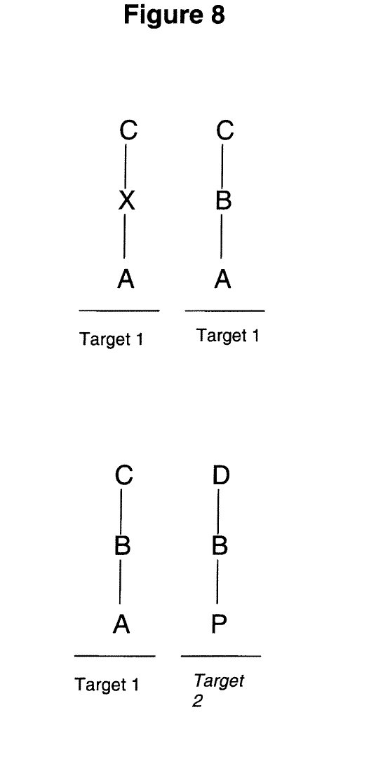

The next parameter for tracking a detection reagent may be which target(s) or detection scheme(s) it is used for. A given reagent, if degenerate, may be used in more than one scheme, such as in the detection of more than one target. (See FIG. 8, lower panel.) Or it may be used only in one scheme. Further, a computer could select the appropriate reagent for an assay from among those that are redundant and hence, interchangeable.

A reagent may accordingly be assigned to a particular target or target panel, to track whether it is used in a test for targets A, B, and/or C, for example. A reagent may also be assigned a further parameter that relates to its function, separate from its level in the overall system. For example, a reagent may be assigned a function as a probe, but could be, as explained above, at level 1 or 2, depending upon the organization of the detection assay.

By using at least one reagent in a set that is redundant, or interchangeable, a particular detection scheme could be automatically modified to choose from more than one set of detection reagents for a given target. This might help to avoid, for example, unwanted interactions between two parallel target detection schemes run on the same sample. A reagent could be substituted with its redundant reagent, in addition, if its stock is running low.

Accordingly, the present application also allows for a method for automatic selection of a set of reagents to detect one or more targets in a sample, wherein the set of reagents comprises at least two layers for detection of a first target and at least two layers for detection of a second target, wherein the set comprises at least one redundant reagent.

In addition, the computer-generated code may include other information about the detection reagent. Examples include the reagent's reaction conditions with the sample (i.e. incubation times and temperatures), any unwanted cross-reactivities it has, the strength of the signal it produces, whether a washing or blocking step should be performed in conjunction, etc.

### Degenerate Reagents and Degenerate Molecular Codes

In some embodiments, at least one member of a reagent set is degenerate such that it can interact with more than one other molecule in the set. Degeneracies may be created by designing the detection reagents to contain two different binding sites. For example, an antigen may contain more than one epitope or a segment of nucleic acid may contain more than one protein binding site or nucleic acid hybridization site.

In some embodiments, one binding site may be itself degenerate, and thus capable of interacting with more than one binding partner. Such a degeneracy may be formed a degenerate molecular code. One example of such a code is a nucleic acid segment or sequence that is capable of specific hybridization to more than one other nucleic acid segment or sequence. Such nucleic acid codes may be generated, for instance, from the use of non-natural bases that form stable base-pairing interactions with more than one other natural or non-natural base. Nucleic acid codes and their associated binding rules may additionally be input into an optional computer or software program, such that the program can determine from the sequences of the nucleic acids what other detection reagents the sequence should interact with.

Non-natural bases that could be used to make a degenerate molecular code may include, for example, purine-like and pyrimidine-like molecules, such as those that may interact using Watson-Crick-type, wobble, or Hoogsteen-type pairing interactions. Examples include generally any nucleobase referred to elsewhere as “non-natural” or as an “analog.”

Examples include: halogen-substituted bases, alkyl-substituted bases, hydroxy-substituted bases, and thiol-substituted bases, as well as 5-propynyl-uracil, 2-thio-5-propynyl-uracil, 5-methylcytosine, isoguanine, isocytosine, pseudoisocytosine, 4-thiouracil, 2-thiouracil and 2-thiothymine, inosine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

Yet other examples include bases in which one amino group with a hydrogen is substituted with a halogen (small “h” below), such as 2-amino-6-“h”-purines, 6-amino-2-“h”-purines, 6-oxo-2-“h”-purines, 2-oxo-4-“h”-pyrimidines, 2-oxo-6-“h”-purines, 4-oxo-2-“h”-pyrimidines. Those will form two hydrogen bond base pairs with non-thiolated and thiolated bases;

respectively, 2,4 dioxo and 4-oxo-2-thioxo pyrimidines, 2,4 dioxo and 2-oxo-4-thioxo pyrimidines, 4-amino-2-oxo and 4-amino-2-thioxo pyrimidines, 6-oxo-2-amino and 6-thioxo-2-amino purines, 2-amino-4-oxo and 2-amino-4-thioxo pyrimidines, and 6-oxo-2-amino and 6-thioxo-2-amino purines.

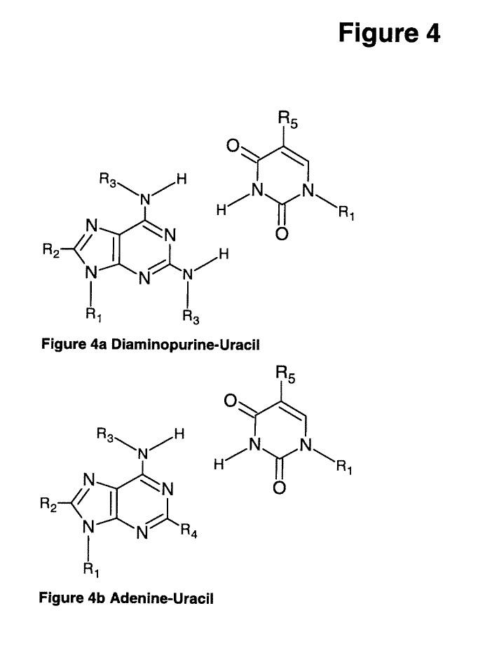

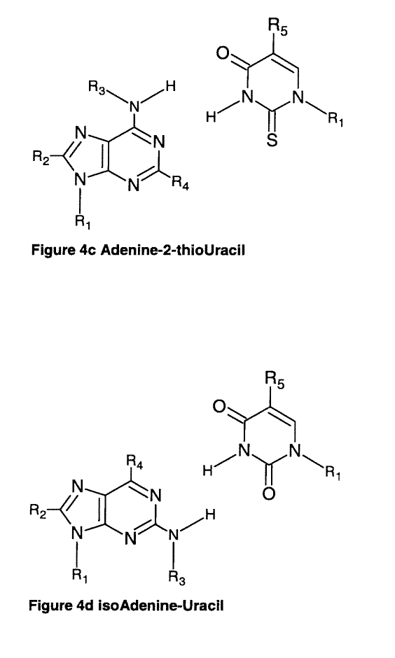

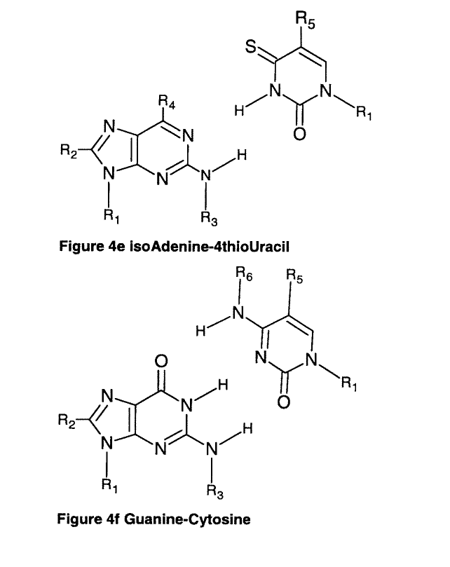

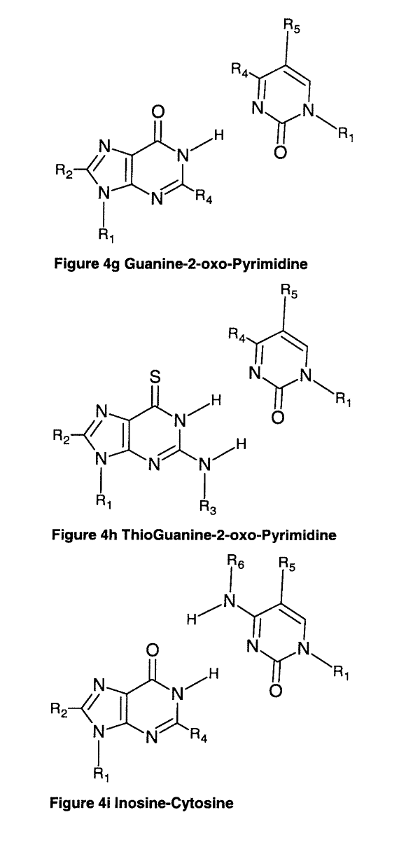

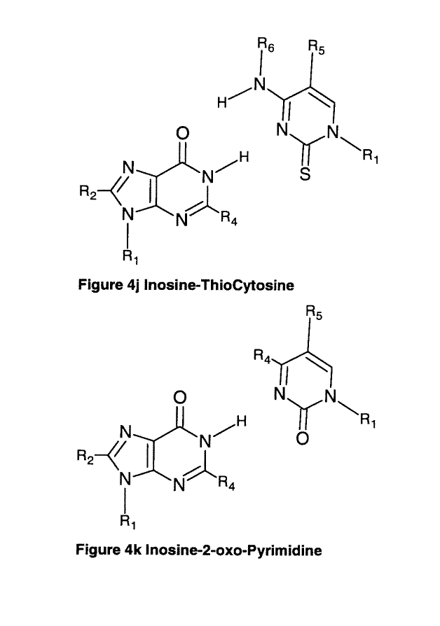

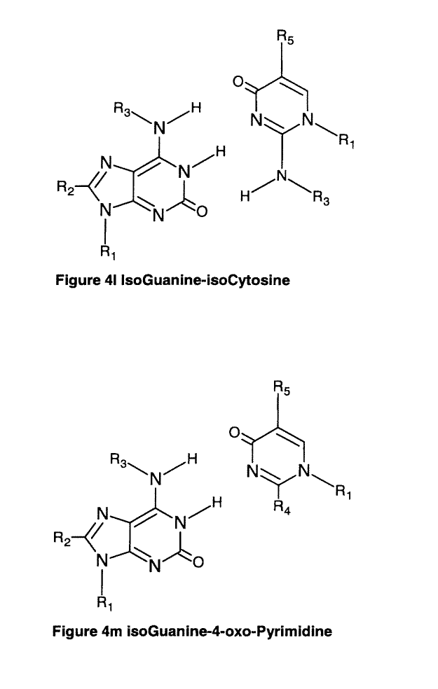

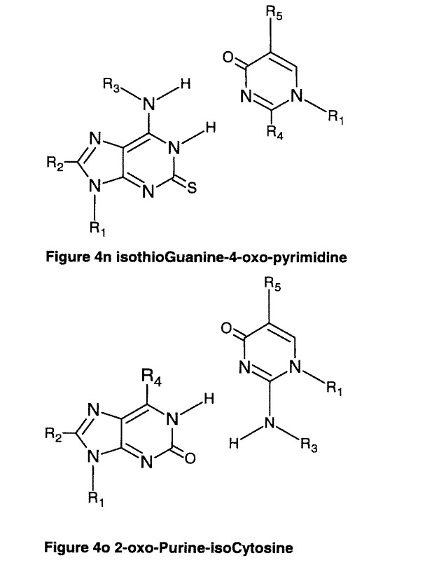

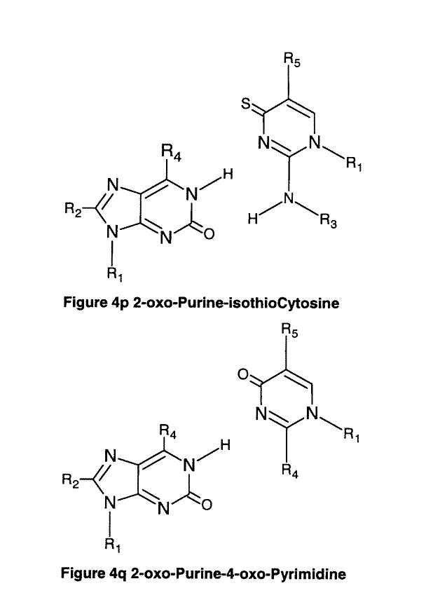

For example, some specific embodiments of non-natural bases are the structures shown in FIG. 4 with the following substituents, which are described in the PCT Application entitled “New Nucleic Acid Base Pairs,” which is incorporated herein by reference.

| Base | | | | | |
|---|---|---|---|---|---|
| (Symbol) | R2 | R3 | R4 | R5 | R6 |
| A | H or CH₃ | H | H | | |
| isoA | H or CH₃ | H | H | | |
| D | H or CH₃ | H | | | |
| G | H or CH₃ | H | | | |
| Gs | H or CH₃ | H | | | |
| I | H or CH₃ | | H | | |
| U | | | | H or CH₃ | |
| U2s | | | | H or CH₃ | |
| U4s | | | | H or CH₃ | |
| C | | | | H or CH₃ | H |
| Py-2o | | | H or CH₃ | H or CH₃ | |
| Cs | | | | H or CH₃ | H |
| isoG | H or CH₃ | H | | | |
| isoGs | H or CH₃ | H | | | |
| Pu-2o | H or CH₃ | | H | | |
| isoC | | H | | H or CH₃ | |
| isoCs | | H | | H or CH₃ | |
| Py-4o | | | H or CH₃ | H or CH₃ | |

| Base | | | | | |
|---|---|---|---|---|---|
| (Symbol) | R2 | R3 | R4 | R5 | R6 |
| A | H or CH₃ | H | CH₃ | | |
| isoA | H or CH₃ | H | CH₃ | | |
| D | H or CH₃ | CH₃ | | | |
| G | H or CH₃ | CH₃ | | | |
| Gs | H or CH₃ | CH₃ | | | |
| I | H or CH₃ | | CH₃ | | |
| U | | | | H or CH₃ | |
| U2s | | | | H or CH₃ | |
| U4s | | | | H or CH₃ | |
| C | | | | H or CH₃ | CH₃ |
| Py-2o | | | H or CH₃ | H or CH₃ | |
| Cs | | | | H or CH₃ | CH₃ |
| isoG | H or CH₃ | CH₃ | | | |
| isoGs | H or CH₃ | CH₃ | | | |
| Pu-2o | H or CH₃ | | CH₃ | | |
| isoC | | | | CH₃ or CH₃ | |
| isoCs | | CH₃ | | CH₃ or CH₃ | |
| Py-4o | | | H or CH₃ | H or CH₃ | |

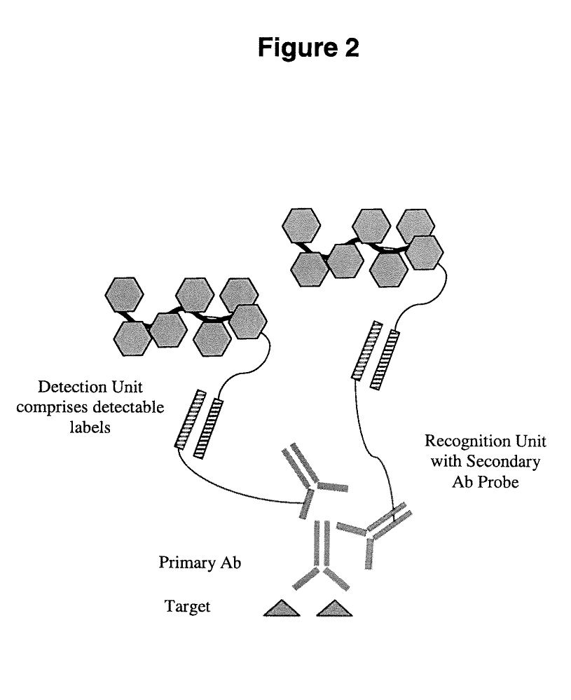

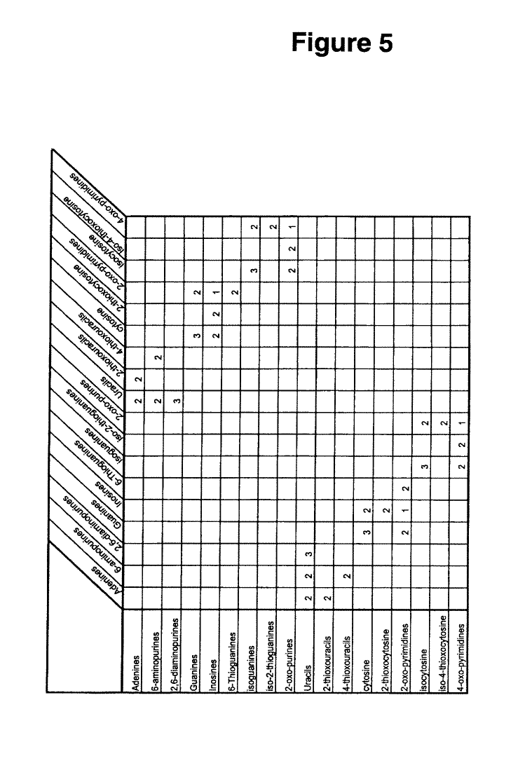

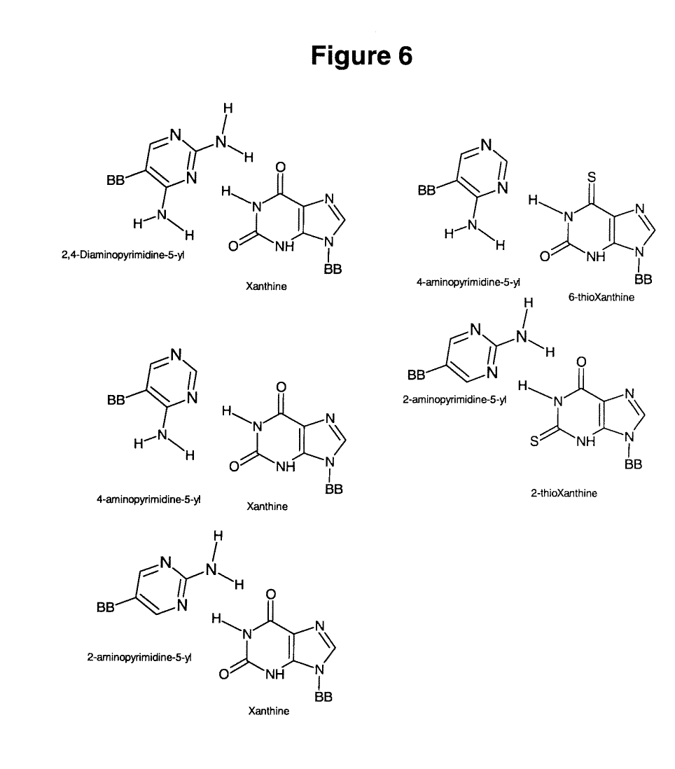

In other examples, one or more of the H or CH₃ are independently substituted with a halogen such as Cl or F. Other example non-natural bases and base-pairs are shown in FIG. 20 herein. R1 in the structures of FIGS. 4-6 may serve as a point of attachment to a backbone group, such as PNA, DNA, RNA, etc. Still other examples are illustrated in FIGS. 2(A) and 2(B) of Buchardt et al. (U.S. Pat. No. 6,357,163).

Non-natural bases such as those exemplified above may be able to form stable base pairing interactions with more than one other base, via 2 or 3-hydrogen bond schemes, for example. The chart below and figures provided herein illustrate several examples of how such degenerate base-pairing schemes lead to the ability to synthesize nucleic acid analog segments with degenerate recognition.

| Base Pairs | Diaminopurine | Adenine |
|---|---|---|
| Uracil | 3 H-bonds | 2 H-bonds |
| 2-ThioUracil | repulsion | 2 H-bonds |
| Base Pairs | Diaminopurine | isoAdenine |
| Uracil | 3 H-bonds | 2 H-bonds |
| 4-ThioUracil | repulsion | 2 H-bonds |
| Base Pairs | Guanine | Inosine |
| Cytosine | 3 H-bonds | 2 H-bonds |
| 2-ThioCytosine | repulsion | 2 H-bonds |
| Base Pairs | Cytosine | 2-oxo-Pyrimidine |
| Guanine | 3 H-bonds | 2 H-bonds |
| ThioGuanine | repulsion | 2 H-bonds |
| Base Pairs | isoGuanine | 2-oxo-Purine |
| isoCytosine | 3 H-bonds | 2 H-bonds |
| isoThioCytosine | repulsion | 2 H-bonds |

In some embodiments, the degenerate molecular codes are nucleic acid analog segments made from DNA or RNA backbones and at least one non-natural base. In other embodiments, they are made from nucleic acids of non-natural backbone units as well. Such non-natural backbone units thus include, but are not limited to, for example, PNA's, LNA's or phosphorothioate or 2′O-methyl nucleosides.

For example, in some embodiments, one or more phosphate oxygens may be replaced by another molecule, such as sulfur. In other embodiments, a different sugar or a sugar analog may be used, for example, one in which a sugar oxygen is replaced by hydrogen or an amine, or an O-methyl. In yet other embodiments, nucleic acid analog segments comprise synthetic molecules that can bind to a nucleic acid or nucleic acid analog. For example, a nucleic acid analog may be comprised of peptide nucleic acids (PNAs), locked nucleic acids (LNAs), or any derivatized form of a nucleic acid. Such backbone units may be attached to any base, including the natural bases A, C, G, T, and U, and non-natural bases.

As used herein, “peptide nucleic acid” or “PNA” means any oligomer or polymer comprising at least one or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 6,201,103, 6,228,982 and 6,357,163; all of which are herein incorporated by reference.

The term PNA also applies to any oligomer or polymer segment comprising one or more subunits of the nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem., 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

As used herein, the term “locked nucleic acid” or “LNA” means an oligomer or polymer comprising at least one or more LNA subunits. As used herein, the term “LNA subunit” means a ribonucleotide containing a methylene bridge that connects the 2′-oxygen of the ribose with the 4′-carbon. See generally, Kurreck, Eur. J. Biochem., 270:1628-44 (2003).

Nucleic acid segments may be synthesized chemically or produced recombinantly in cells (see e.g. Sambrook et al. (1989) Molecular Cloning: A Laboratory Manual, 2nd ed. Cold Spring Harbor Press). Methods of making PNAs and LNAs are also known in the art (see e.g. Nielson, 2001, Current Opinion in Biotechnology 12:16; Sorenson et al. 2003, Chem. Commun. 7(17):2130).

Different nucleic acid analog segments may hybridize, for instance, using Watson-Crick-type, wobble, or Hoogsteen-type base-pairing. Accordingly, the nucleic acid analog segments comprise sequences which allow for hybridization to take place at a desired stringency.

In other embodiments of the invention, degenerate molecular codes may be created from other types of molecular interactions. For instance, a given antigen epitope or a given substrate may be recognized by more than one antibody or enzyme. (See, for example, FIG. 7.)

### Systems and Methods for Detection

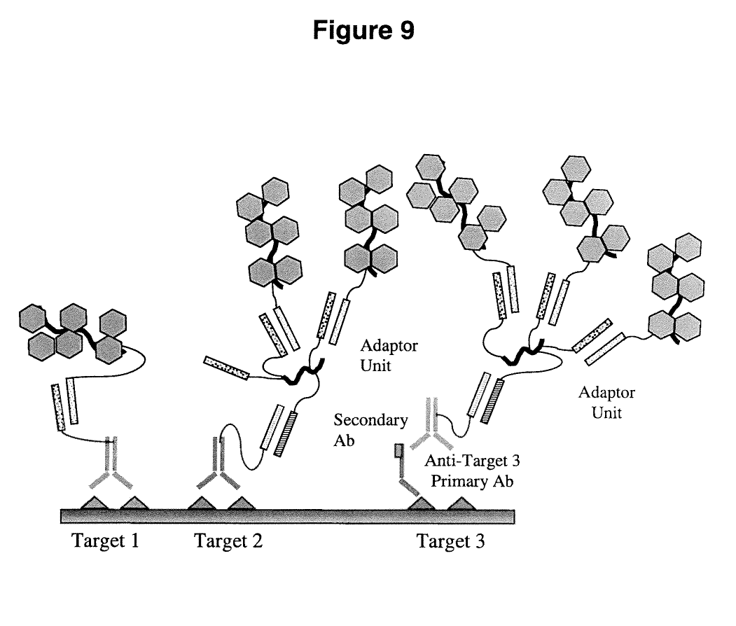

Some embodiments of this invention are based on the principle of using the automatically selected reagent sets as part of a system of detection reagents for one or more targets. Such redundant, and optionally, degenerate detection reagents may, for instance, allow for a degree of interchangeability from one detection protocol to another. (See FIGS. 8-9.) For instance, in some embodiments of the invention, a molecular entity carrying a detectable label specifically interacts with a probe or with another molecular entity comprising the probe.

For example, if the probe is degenerate, that probe could in turn interact with more than one type of detectable label. Such interactions would then allow the experimenter to choose a suitable label for the experiment from among those the probe specifically recognizes, allowing for greater flexibility. Accordingly, the two interchangeable detectable labels become redundant reagents in the system. In other cases, should supplies of one type of label run low, a different adaptor or label could be substituted without adversely affecting the detection assay.

In other examples, a degenerate probe could specifically bind to more than one type of adaptor molecular entity, allowing the adaptors to be redundant. If the different adaptors interact with different detectable labels or molecular entities carrying those labels, then the method of labeling could similarly be expanded. In other embodiments, the detectable label or molecular entity carrying the label could be degenerate, such that it could be used to bind specifically to more than one type of probe or adaptor. In yet other embodiments, the adaptor could be degenerate, allowing it to link together more than one probe-detectable label combination. (An example is shown in the bottom panel of FIG. 8.)

The present invention also contemplates a system for detecting one or more targets in a sample, comprising at least one set of detection reagents for each target, each set comprising at least two detection reagents, wherein at least one detection reagent in the system is degenerate, and optionally comprises a degenerate molecular code, and wherein, optionally, each container for each detection reagent comprises a computer-generated code, wherein the degenerate molecular code and the computer-generated code allow each set of detection reagents to associate with its intended target.

In some systems, the degenerate reagents could be made from nucleic acids, such as a nucleic acid analog segment that specifically hybridize to more than one other nucleic acid segment, or two different nucleic acid segments, each with different binding properties. In some embodiments, the nucleic acid is PNA or LNA rather than DNA or RNA. (See, e.g., FIGS. 4-6.) In other systems, the degenerate reagent comprises at least one hapten or at least one antigen. For example, an antigen may be specifically recognized by more than one antibody, either because it contains one epitope with multiple binding partners or because it contains two or more different epitopes. (See, e.g., FIG. 7.) Such systems may be used to detect, for example, protein targets, DNA targets, and RNA targets in a sample, as well as other molecules or entities such as carbohydrates, membrane lipids, chemical toxins, and the like.

Such systems may be used to in manual or automated detection assay protocols. The computer-generated codes may be helpful in assigning and moving reagents in an automated process, for example. In either case, the systems may be used within detection apparatuses. Such apparatuses may, for instance, serve to dispense reagents in appropriate amounts, apply washing steps, hold the samples, incubate the samples at appropriate temperatures during a detection process, and detect signal.

Some apparatuses may employ computer hardware and software to control the progress of the detection method, such as the dispensing of reagents. For instance, computer software could be used to select the set of reagents, and also to move and dispense reagent containers for a particular detection method at the appropriate time. In such instances, a computer-generated code on the containers may be helpful in controlling the apparatus.

In some systems, the computer-generated code comprises a bar code, or another type of known coding for tracking the use or movement of containers, such as a sku code. Even simple numbering systems, colors, or other computer-detectable codes may suffice. In some embodiments, the computer-generated code could include information about the function and level of the detection reagents and their interactions with each other, as well as information such as how long and at what temperature they should be incubated with the sample, what activity or signal levels they have, what unwanted cross-reactivities they show, and whether blocking or washing steps should be performed.

The instant invention also involves detection methods, for instance, A method of detecting one or more targets in a sample, comprising

(a) obtaining a sample potentially comprising one or more targets;
(b) automatically selecting a set of reagents for detection of the one or more targets,

(i) wherein one of the reagents in the set is redundant to another reagent in the set, and the reagents comprise at least two layers for detection of each of the two targets;

(c) contacting the sample with the set of detection reagents;
(d) detecting the presence or absence of signals from the association of the sets of detection reagents with the one or more targets; and
(e) correlating the presence or absence of the signals with the presence or absence of the one or more targets in the sample.

In some embodiments, at least one reagent in the set is degenerate. For instance, that reagent may contain a degenerate molecular code, such as a nucleic acid code comprising at least one non-natural nucleic acid base, as described above. In others, the degeneracy could be formed from other types of interactions, such as antigen-antibody interactions or other protein-ligand interactions.

In some embodiments, two or more targets are detected.

In some embodiments, two or more targets are detected. The reagent sets and methods of the invention may be used to detect, for example, protein targets, DNA targets, and RNA targets in a sample, as well as other molecules or entities such as carbohydrates, membrane lipids, chemical toxins, and the like.

In some methods, a computer-generated code is used for the automated selection of the reagent set. That code may, for instance, help to determine whether the redundant reagent should be substituted in the detection assay for its interchangeable partner. The computer-generated code may also help to organize the dispensing and control of the various reagents by their properties, such as what layer or function in the detection scheme they have, which target detection they belong to, what redundancies and/or degeneracies they have, etc.

In some detection methods, the level of the target in the sample may be detected qualitatively. For instance, in some cases the experimenter is interested only in the presence or absence of a target. However, in other cases, the experimenter may wish to detect the target quantitatively, to also determine its relative concentration or amount in the sample. In such cases, methods such as densitometry could be employed to convert the signal from a target into a quantitative or digital reading. If a computer-associated apparatus is used, the apparatus and software could be adapted to quantitatively read the signal generated from the detection method. Alternatively, a separate densitometry apparatus and program could be employed.

### Automated Selection of Staining Schemes and Reagent Sets

The automated selection of a reagent set may optionally also include optimizing various parameters such as the overall time of the reaction or the signal intensity that results. The user may also prefer certain stains over other available choices. The available quantity of a given reagent may also be a factor to consider. Hence, a computer-generated code may include information related to those user preferences so that a given software algorithm may select an optimized set of reagents. The coded data can be both general for the particular reagent and lot dependant, for example including:

Specific binding pattern with different species

Unwanted cross reactivity with different species

Blocking reagents

Reaction kinetics at different temperatures and dilutions

Amplification power

Activity as function of age

as well as more traditional data, such as:

Reagent Name

Lot number

Production date

Storage conditions

Expiry

Volume.

Multi-parameter coding may also assist in ensuring the consistency and reproducibility of a diagnostic assay, such as one performed according to an approved regulatory protocol for diagnosing disease. In some embodiments, it may thus be helpful to effectively “lock” the system so that the user must allow automated selection of the reagents and optionally, the detection protocol. Further, when coding multiple parameters, only a computer may be able to interpret the information in a reasonable manner. In other embodiments, the system may be “open” or “partially open” such that the general user is aware of how the set of reagents is being selected and can amend the selection procedure if needed.

### WORKING EXAMPLES

### Example 1

### Coded Detection Reagents

Assume a goat anti-mouse HRP polymeric conjugate gives a 6-fold amplification compared to a monomeric conjugate, at reaches saturation after 10 min incubation at 20° C. or after 4 minutes at 25° C. Further assume that 4-fold dilution of the antibody reagent has the same effect as doubling the incubation time. Further assume that the reagent containing goat IgG, has an HRP enzyme with moderate enzyme activity, is specific against mouse IgG, has 2% cross reactivity against rabbit IgG, 15% cross reactivity against rat IgG, contains BSA, gives 5% background after 2 wash cycles and 2% background after 3 wash cycles, and that the reagent's enzymatic activity drops by 5% each month after the production date.

That information could be represented in the form of a code such as: 6-10.20.4.25-4.2-G-HM-M-2R-15Ra-B-5.2.2.3-5.

A similar code could be designed to incorporate information about interacting nucleic acid segments as well. An example could be a similar polymeric reagent containing alkaline phosphatase (AP) and two binding entities with sequence TCD-DGsGs-TAC-A and CAT-DGsD-ATC-Gs. Those binding entities could be made from DNA, or a non-natural backbone such as PNA, for example. The binding pattern would be specific against UsGUs-DPP-TTG-D and PGD-UsTP-TDUs-G, respectively, for example.

If the reagent gives 4-fold amplification compared to a monomeric conjugate; reaches saturation after 8 min at 20° C. and after 2 minutes after 25° C.; if 5-fold dilution of the reagent has the same effect as doubling the incubation time; the reagent contains AP enzyme with a high enzyme activity; contains BSA, gives 4% background after 2 wash cycles and 2% after 3 wash cycles; and if the activity drops by 3% per months from production date, then a code to represent the reagent could be as follows: 4-8.20.2.25-5.2-TCDDGsGsTACA-CATDGsDATCGs-AH-B-4.2.2.3-3.

In the code above, each number or letter represents a piece of information about the reagent, as provided above.

### Example 2

### Exemplary Reagent Systems

An example of a reagent system based on conventional reagents in a ready to use (RTU) format may be as follows:

### Group 1: (Primary Reagents)

A FITC-labeled mouse antibody against Human ER protein

### Group 2: (Amplification Reagents)

B Rabbit anti-FITC
C Polymeric goat anti-mouse antibody
D FITC-labeled rabbit anti-mouse antibody

### Group 3: (Enzyme Conjugates)

E Polymeric goat anti-rabbit HRP conjugate
F Polymeric rabbit anti-goat HRP conjugate
G Polymeric goat anti-mouse HRP conjugate

### Group 4: (Detectable Labels)

H DAB chromogen

Polymeric conjugates give about 5-10 times stronger signals than conventional reagents. Thus, those reagents require half the incubation time of conventional reagents. A DAB staining of the ER protein could be done by one of the following five protocols below, each giving approximately the same staining results. The letters represent the addition of the successive reagents above and the incubation time after each addition.

Protocol #1: A (10 min), B (10 minutes), E (10 minutes), H (5 minutes)
Protocol #2: A (20 minutes), G (20 minutes), H (5 minutes)
Protocol #3: A (5 minutes), C (5 minutes), F (5 minutes), H (5 minutes)
Protocol #4: A (5 minutes), D (10 minutes), E (10 minutes), H (5 minutes)
Protocol #5: A (5 minutes), D (5 minutes), B (5 minutes), E (5 minutes), H (5 minutes)

Optionally, temperature variables may also be included. The protocols do not include optional washing and blocking steps, for simplicity.

The protocols may start with the same primary reagent and same chromogen, but use different intervening reagents, and hence, different steps. Protocol #2 have the fewest steps and is the longest, whereas protocol #5 uses more steps and has a shorter incubation time. Reagent E is used in step 3, while D is used in step 2 of the protocol, respectively. If both visualization reagents were not available in the system due to a low volume or due to instrument scheduler constraints, protocol #2 and #3 could still be performed. The software could accordingly help the user to select the best overall staining scheme, given the time constraints and available reagents. Each reagent above may further be coded as described in Example 1.

### Example 3

### Additional Exemplary Reagent Systems

A reagent system based on reagents in a ready to use (RTU) format is as follows:

### Group 0: (Target Retrieval)

Weak target retrieval solution (e.g. citrate pH 6)
II Medium-strong target retrieval solution (Tris/EDTA, pH 8)
III Strong target retrieval solution (e.g. Tris/EDTA, pH 9)

### Group 1: (Recognition Unit)

A Mouse antibody against Human ER protein conjugated to nucleic acid analog sequences: TTT-UsUsUs and TCD-DGsGs-TAC-A (“Anna”)
B Mouse antibody against Human HER2 protein conjugated to: CAT-DGsD-ATC-Gs (“Erna”)

### Group 2: (Amplification)

C Molecular entity comprising at least one sequence: UsGUs-DPP-TTG-D (“Alex”) and at least one sequence: UsUsUs-TTT
D Molecular entity comprising at least one sequence: PGD-UsTP-TDUs-G (“Elmer”) and at least one sequence: TTT-UsUsUs

### Group 3: (Enzyme Conjugate)

E Polymeric HRP conjugate with the nucleic acid sequence: AAA-AAA-AAA
F Polymeric HRP conjugate with PGD-UsTP-TDUs-G (“Elmer”)

### Group 4: (Detectable Label)

H Diaminobenzidine (DAB HRPD) chromogen
I Liquid fast red (LFR AP) chromogen

Note: CAT-DGsD-ATC-Gs (“Erna”) specifically hybridizes to PGD-UsTP-TDUs-G (“Elmer”), while TCD-DGsGs-TAC-A (“Anna”) specifically hybridizes to UsGUs-DPP-TTG-D (“Alex”). The sequence AAA-AAA-AAA specifically hybridizes to UsUsUs-TTT and TTT-UsUsUs, whereas UsUsUs-TTT and TTT-UsUsUs do not bind to each other.

A DAB staining of the ER protein is carried out using one of the protocols below, which, for simplicity, do not include optional washing and endogen peroxidase blocking steps. The steps below list the reagent to be added and the incubation time. Coding to change or control temperature may also be included.

Protocol #1: III, A (10 min), E (5 minutes), H (5 minutes)
Protocol #2: I, A (5 minutes), C (5 minutes), E (5 minutes), H (5 minutes)

The second protocol gives higher amplification to compensate for the weaker target retrieval, but nevertheless, still allows for a relatively short incubation time.

A DAB staining of the HER2 protein is carried out with:

Protocol #3: B (10 min), D (5 minutes), E (5 minutes), H (5 minutes)
If one wanted a red LFR/AP staining instead, the protocol would be:
Protocol #4: B (10 min), F (10 minutes), I (5 minutes)

A double staining of both targets on the same sample is performed as shown below, resulting in a DAB/HRP-stained ER target and a LFR/AP-stained HER2 target.

Protocol #4: A and B (10 min), E and F, H (10 minutes), I (5 minutes).
Alternatively, to DAB/HRP stain both targets, the protocol could be:
Protocol #5: A and B (10 min), D (10 minutes), E (5 minutes), H (5 minutes).

Accordingly, a set of reagents in the system depicted in this example allows for multiple types of staining protocols, because some of the reagents have degenerate binding patterns.

### Example 4

### Preparation and Use of Detection Reagents and Systems Using Molecular Entities Interacting Through Nucleic Acid Base-Pairs

### Example 4a

### Preparation of Pyrimidinone-Monomer

1. In dry equipment 4.6 g of solid Na in small pieces was added to 400 mL ethanol (99.9%), and was dissolved by stirring. Hydroxypyrimidine hydrochloride, 13.2 g, was added and the mixture refluxed for 10 minutes. Then 12.2 mL ethyl-bromoacetate (98%) was added and the mixture refluxed for 1½ hour. The reaction was followed using Thin Layer Chromatography (TLC). The ethanol was evaporated leaving a white compound, which was dissolved in a mixture of 80 mL of 1M NaCitrate (pH 4.5) and 40 mL of 2M NaOH. This solution was extracted four times with 100 mL Dichloromethane (DCM). The DCM phases were pooled and washed with 10 mL NaCitrate/NaOH— mixture. The washed DCM phases were evaporated under reduced pressure and resulted in 17.2 g of crude solid product. This crude solid product was recrystallized with ethylacetate giving a yellow powder. The yield for this step was 11.45 g (63%).

2. The yellow powder, 12.45 g. from above was hydrolyzed by refluxing overnight in a mixture of 36 mL DIPEA, 72 mL water and 72 mL dioxane. The solvent was evaporated and water was removed from the residue by evaporation from toluene. The yield for this step was 100%.

3. OBS. Pyrimidinone acetic acid (10.5 g), 16.8 g PNA-backbone ethylester, 12.3 g DHBT-OH, 19 mL Triethylamine was dissolved in 50 mL N,N-dimethylformamide (DMF). DIPIDIC (11.8 mL) was added and the mixture stirred overnight at room temperature. The product was taken up in 100 mL DCM and extracted three times with 100 mL of dilute aqueous NaHCO₃. The organic phase was extracted twice with a mixture of 80 mL of 1M NaCitrate and 20 mL of 4M HCl. Because TLC showed that some material was in the citrate phase, it was extracted twice with DCM. The organic phases were pooled and evaporated. Because there was a precipitation of urea, the product was dissolved in a DCM, and the urea filtered off. Subsequent evaporation left an orange oil. Purification of the orange oil was performed on a silica column with 10% methanol in DCM. The fractions were collected and evaporated giving a yellow foam. The yield for this step was 7.0 g (26.8%).

4. The yellow foam (8.0 g) was hydrolyzed by reflux overnight in 11 mL DIPEA, 22 mL water, and 22 mL dioxane. The solvent was evaporated and the oil was dehydrated by evaporation from toluene leaving an orange foam. The yield for this step was 100%.

### Example 4b

### Second Method of Preparing Pyrimidonone Monomer

Step 1. In dry equipment 9.2 g of solid Na in small pieces was dissolved in 400 mL ethanol (99.9%), with stirring. Hydroxypyrimidine hydrochloride, 26.5 g, was added, and the mixture was stirred for 10 minutes at 50° C. Then 24.4 mL Ethyl bromoacetate (98%) was added and the mixture stirred at 50° C. for 1 hour. The reaction was followed using Thin Layer Chromatography (TLC).

The ethanol was evaporated leaving a white compound, which was dissolved in 70 mL of water and extracted with 20 mL DCM. Another 30 mL of water was added to the water phase, which was extracted with 3×100 mL DCM. The DCM-phase from the first extraction contains a lot of product, but also some impurities, wherefore this phase was extracted twice with water. These two water phases then were back extracted with DCM.

The combined DCM phases were pooled and washed with 10 mL water. The washed DCM phases were evaporation under reduced pressure and resulted in 25.1 g yellow powder. The yield for this step was 25.1 g=69%. Maldi-Tof: 181.7 (calc. 182).

Step 2. 34.86 g yellow powder from above was dissolved in 144 mL 2M NaOH. After stirring 10 minutes at room temperature, the mixture was cooled in an ice bath. Now 72 mL 4 M HCl (cold) was added. The product precipitated. After stirring for 5 minutes, the precipitate was filtered and thoroughly washed with ice water. Drying in a dessicator under reduced pressure left 18.98 g yellow powder. The yield for this step was 18.98 g=64%.

Step 3. Pyrimidinone acetic acid 11.1 g and triethylamine 12.5 mL were dissolved in N,N-dimethylformamide (DMF) 24 ml, HBTU 26.2 g was added plus 6 mL extra DMF. After 2 minutes a solution of PNA-Backbone ethylester 14.7 g dissolved in 15 mL DMF was added. The reaction mixture was stirred at room temperature and followed using TLC. After 1½ hour precipitate had formed. This was filtered off.

The product was taken up in 100 mL DCM and extracted with 2×100 mL dilute aqueous NaHCO3. Both of the aqueous phases were washed with a little DCM. The organic phases were pooled and evaporated. Evaporation left an orange oil. Purification of the product was done on a silica column with 10-20% methanol in ethylacetate. The fractions were collected and evaporated giving a yellow oil. The oil was dissolved and evaporated twice from ethanol. The yield from this step was 20.68 g=90%.

Step 4. The yellow oil (18.75 g) was dissolved in 368 mL 0.2 M Ba(OH)₂. Stirring for 10 minutes before 333 mL 0.221 M H2SO4 was added. A precipitation was performed immediately. Filtration through cellite, which was washed with water. The solvent was evaporated. Before the evaporation was at end, the product was centrifuged to get rid of the very rest of the precipitation. Re-evaporation of the solvent left a yellow oil. The yield from this step was 13.56 g=78%.

Step 5. To make a test on the P-monomer 3 consecutive P's were coupled to Boc-L300-Lys(Fmoc) resin, following normal PNA standard procedure. The product was cleaved from the resin and precipitated also following standard procedures: HPPP-L300-Lys(Fmoc). Maldi-Tof on the crude product: 6000 (calc. 6000) showing only minor impurities.

### Example 4c

### Preparation of the Thio-Guanine Monomer

1. 6-Chloroguanine (4.93 g) and 10.05 g K₂CO₃ was stirred with 40 mL DMF for 10 minutes at room temperature. The reaction mixture was placed in a water bath at room temperature and 3.55 mL ethyl bromoacetate was added. The mixture was stirred in a water bath until TLC (20% Methanol/DCM) showed that the reaction was finished. The precipitated carbonate was filtered off and washed twice with 10 mL DMF. The solution, which was a little cloudy, was added to 300 ml water, whereby it became clear. On an ice bath the target compound slowly precipitated. After filtration the crystals were washed with cold ethanol and dried in a desiccator. The yield for this step was 3.3 g (44.3%) of ethyl chloroguanine acetate.

2. Ethyl chloroguanine acetate (3.3 g) was dissolved by reflux in 50 mL absolute ethanol. Thiourea (1.08 g) was added. After a refluxing for a short time, precipitate slowly began forming. According to TLC (20% Methanol/DCM) the reaction was finished in 45 minutes. Upon completion, the mixture was cooled on an ice bath. The precipitate was then filtered and dried overnight in a desiccator. The yield for this step was 2.0 g (60%) ethyl thioguanine acetate.

3. Ethyl thioguanine acetate (3.57 g) was dissolved in 42 mL DMF. Benzylbromide (2.46 mL) was then added and the mixture stirred in an oil bath at 45° C. The reaction was followed using TLC (25% Methanol/DCM). After 3 hours all basis material was consumed. The step 3 target compound precipitated upon evaporation under reduced pressure and high temperature. The precipitate was recrystallized in absolute ethanol, filtered and then dried in a desiccator. The yield for this step was 3.88 g (82%) of methyl benzyl thioguanine ethylester.

4. Methyl benzyl thioguanine ethylester (5.68 g) was dissolved in 12.4 mL of 2M NaOH and 40 mL THF, and then stirred for 10 minutes. The THF was evaporated by. This was repeated. The material was dissolved in water and then 6.2 mL of 4M HCl was added, whereby the target product precipitated. Filtering and drying in a desiccator. The yield for this step was 4.02 g (77%).

5. The product of step 4 (4.02 g), 3.45 g backbone ethylester, 9 mL DMF, 3 mL pyridine, 2.1 mL triethylamine and 7.28 g PyBop were mixed and then stirred at room temperature. After 90 minutes a solid precipitation formed. The product was taken up in 125 mL DCM and 25 mL methanol. This solution was then extracted, first with a mixture of 80 mL of 1M NaCitrate and 20 mL of 4M HCl, and then with 100 mL dilute aqueous NaHCO₃. Evaporation of the organic phase gave a solid material. The material was dissolved in 175 mL boiling ethanol. The volume of the solution was reduced to about 100 mL by boiling. Upon cooling in an ice bath, the target product precipitate. The crystals were filtered, washed with cold ethanol and then dried in a desiccator. The yield of this step was 6.0 g (86%.)

6. The product of step 5 (6.0 g) was dissolved in 80 mL THF, 7.5 mL 2M NaOH and 25 mL water. The solution became clear after ten minutes of stirring. THF was evaporated. Water (50 mL) was added to the mixture. THF was evaporated. Water (50 mL) was added to the mixture. When the pH was adjusted by the addition of 3.75 mL of 4M HCl, thio-guanine monomer precipitated. It was then filtered, washed with water and dried in a desiccator. The yield for this step was 5.15 g (91%).

### Example 4d

### Preparation of Diaminopurine Acetic Acid Ethyl Ester

1. Diaminopurine (10 g) and 40 g of K₂CO₃ were added to 85 mL of DMF and stirred for 30 minutes. The mixture was cooled in a water bath to 15° C. Ethyl bromoacetate (3 mL) was added three times with 20 minute intervals between each addition. This mixture was then stirred for 20 minutes at 15° C. The mixture was left in the water bath for another 75 minutes, and the temperature increased to 18° C. The DMF was removed by filtering and the remaining K₂CO₃ was added to 100 mL of ethanol and refluxed for 5 minutes. Filtering and repeated reflux of the K₂CO₃ in 50 mL ethanol, filtering. The pooled ethanol phases were placed in a freezer, after which crystals formed. These crystals were filtered, washed with cold ethanol, filtered again and then dried in a desiccator overnight. The yield for this step was 12 g (76%).

### Example 4e

### Preparation of an L₃₀-Linker to Connect Elements of a Multicomponent Molecular Entity

This linker is one example of a linking element that may covalently bridge elements of a polymeric molecular entity. Multiple L₃₀ linker segments may be strung together to create a longer linker, if desired.

1. A solution of 146 mL of 2,2′-(Ethylenedioxy)bis(ethylamine) (98%) in 360 mL of THF was cooled in an ice bath. Di-tert-butyl dicarbonate (97%) (65 g) in 260 mL THF was added dropwise over one hour. The solvent was evaporated. The remaining oil was dissolved in water and then evaporated off. The oily product was dissolved in 300 mL water, extracted with 300 mL DCM, then washed twice with 150 mL of DCM. The collected organic phase was washed with 50 mL of water before evaporating to about half the volume. The organic phase was then extracted with 400 mL of 1M NaCitrate (pH 4.5), and then extracted again with 50 mL of 1M NaCitrate (pH 4.5). The aqueous phases were washed with 50 mL DCM before cooling on an ice bath. While stirring, 100 mL of 10M NaOH was added to the aqueous washed aqueous phases resulting in pH of 13-14. In a separation funnel the product separated on its own. It was shaken with 300 mL DCM and 50 ml water. The organic phase was evaporated, yielding a white oil. The yield for this step was 48.9 g (65.7%). The product had a predicted molecular formula of C₁₁H₂₄N₂O₄ (MW 248.3).

2. Boc-amine (76.2 g) was dissolved in 155 mL pyridine. Diglycolic anhydride (54.0 g) (90%) was added. After stirring for 15 minutes the intermediate product separated out and then 117 mL Acetic Anhydride (min. 98%) was added and the mixture stirred at 95° C. for 1 hour. The solution was then put under reduced pressure and evaporated. Water (117 mL) was added, and the mixture was then stirred for 15 minutes, after which 272 mL of water and 193 mL of DCM were added. The organic layer was extracted twice with 193 mL of 1M Na₂CO₃ and then twice with a mixture of 72 mL of 4M HCl and 289 mL of 1M NaCitrate. After each extraction the aqueous phase was washed with a little DCM. The collected organic phase was washed with 150 mL of water. The solvent was evaporated leaving the product as an orange oil. This yield for this step was 100.3 g (0.29 mol) (94%). The product had a predicted molecular formula of C₁₅H₂₆N₂O₇ (MW 346.4).

3. The product from step 2 (100.3 g) was dissolved in an equal amount of THF and was then added dropwise to 169.4 mL of 2,2′-(Ethylendioxy)bis(ethylamine) at 60° C. over the period of 1 hour. The amine was distilled from the reaction mixture at 75-80° C. and a pressure of 3×10⁻¹ mBar. The residue from the distillation was taken up in a mixture of 88 mL of 4M HCl and 350 mL of 1M NaCitrate and then extracted three times with 175 mL of DCM. The aqueous phase was cooled in an ice bath and was cautiously added to 105 mL of 10M NaOH while stirring. In a separation funnel the product slowly separated from the solution. When separated 100 mL of water and 950 mL of DCM were added to the product. Stirring for some minutes before pouring to a separation funnel. The pH in the aqueous phase should be 14. The aqueous phase was extracted four times with 150 mL of DCM. The solvent was evaporated. The oily residue was dehydrated by evaporation from toluene, giving a yellow oil. The yield for this step was 115.48 g (81%). The product had a predicted molecular formula of C₂₁H₄₂N₄O₉ (MW 494.6).

4. The Boc-amine (115.48 g) from step 3 was dissolved in 115 mL of pyridine. Diglycolic anhydride (40.6 g) (90%) was added and the mixture stirred for 15 minutes, after which the intermediate product came out. Acetic Anhydride (97 mL) (min. 98%) was added and the mixture stirred at 95° C. for 1 hour. The mixture was then evaporated under reduced pressure. The mixture was then cooled and then 80 mL of water was added. This mixture was stirred for 15 minutes and then 200 mL of water and 150 mL of DCM were added. The organic layer was extracted twice with 150 mL of 1M Na₂CO₃ and then twice with a mixture of 53 mL of 4M HCl and 213 mL of 1M NaCitrate. After each extraction the aqueous phase was washed with a little DCM. The collected organic phase was washed with 150 mL of water. The solvent was evaporated. The oily residue was dehydrated by evaporation from toluene, giving a yellow oil. The yield for this step was 125 g (92%). The product had a predicted molecular formula of C₂₅H₄₄N₄O₁₂ (MW 592.6), with a mass spectrometry determined molecular weight of 492.5.

Further purifying of the product could be done on a silica column with a gradient from 5-10% methanol in DCM. The yield from the column purification was 69% and produced a white oil.

5. White oil (12.4 g) from step 4 was dissolved in a mixture of 12 mL water and 12 mL 1,4-Dioxane (99%) and was then heated to reflux. DIPEA (6 mL) was added and refluxed for 30 minutes. This mixture was cooled and then evaporated. The oily residue was dehydrated by evaporation from toluene, giving a yellow oil. The product had a predicted molecular formula of C₂₅H₄₆N₄O₁₄ (MW 610.6).

### Example 4f

### Exemplary Embodiments of PNA Sequences

All are made by PNA standard procedures are described below.

TABLE 1

| SEQUENCE | | N- | C- | MOLECULAR |
|---|---|---|---|---|
| DESIGNATION | PNA SEQUENCES¹ | TERMINAL | TERMINAL | WEIGHT |
| SEQ. AA | TCD-DGₛGₛ-TAC-A | FLU-L₃₀- | -LYS(CYS) | 8805 |
| SEQ. AB | UₛGUₛ-DPP-TTG-D | FLU-L₃₀- | -LYS(CYS) | 8727 |
| SEQ. AC | CUₛGₛ-GₛDD-TUₛD-GₛDC | FLU-L₃₀- | -LYS(CYS) | 9413 |
| SEQ. AD | GTP-TAA-TTP-PAG | FLU-L₃₀- | -LYS(CYS) | 9203 |
| SEQ. AE | DGₛT-CGₛD-DGₛG-UₛCUₛ | FLU-L₃₀- | -LYS(CYS) | 9413 |
| SEQ. AF | AGA-CPT-TPG-APT | FLU-L₃₀- | -LYS(CYS) | 9187 |
| SEQ. AG | TCD-DI I-TAC-A | FLU-L₃₀- | -LYS(CYS) | 8742 |

¹Flu is fluorescein; T is thiamine; C is cytosine; D is diaminopurine; Gₛ is thioguanine; A is Adenine; Uₛ is 2/4-thiouracil; G is guanine; P is pyrimidone; I is inosine.

### Example 4g

### Three PNAs with the L₃₀ linker with different amino acids at the C-terminal

BA: Flu-L₃₀-DGT-DTC-GTD-CCG-Lys(Acetyl)
BB: Flu-L₃₀-DGT-DTC-GTD-CCG-Lys(Cys)
BC: Flu-L₃₀-DGT-DTC-GTD-CCG-Lys(Lys)₃

### Example 4h

### Synthesis of Flu-L₉₀-Lys(Flu)-L₃₀-Lys(Cys)

Using standard procedures provided below, an MBHA-resin was loaded with Boc-Lys(Dde)-OH. Using a peptide synthesizer, amino acids were coupled according to PNA solid phase procedure provided in Example 18d yielding Boc-L₉₀-Lys(Fmoc)-L₃₀-Lys(Dde). The Boc and Fmoc protections groups were removed and the amino groups marked with flourescein using the procedure in Example 18e. Then, the Dde protection group was removed and 0.4 M cysteine was added according to the procedure in Example 18b. The PNA was cleaved from the resin, precipitated with ether and purified on HPLC according to Example 18d. The product was found to have a molecular weight of 3062 using MALDI-TOF mass spectrometry; the calculated molecular weight is 3061.

### Example 4i

### Synthesis of a Conjugate Made from Sequence AA from Example 5, DexVS70, and Flu(10)

Dextran (with a molecular weight of 70 kDa) was activated with divinylsulfone to a degree of 92 reactive groups/dextran polymer; this product is designated DexVS70.

| | | |
|---|---|---|
| 280 μL DexVS70 | 20 | nmol |
| 66 μL Flu₂Cys | 160 | nmol (prepared from Example above) |
| 25 μL 0.8 M NaHCO₃ | | |
| pH = 9.5 | | |
| 29 μL H₂O | | |

The above four compounds were mixed. The mixture was placed in a water bath at 30° C. for 16 hours. The mixture was added to 50 nmol of freeze-dried PNA (sequence AA from Example above). The mixture was placed in a water bath at 30° C. for 30 minutes. The conjugating reaction was quenched with 50 μL of 500 mM cysteine for 30 minutes at 30° C. Purification of the product was performed using FPLC: column SUPERDEX®—200, buffer 10 mM Hepes 100 mM NaCl, method 7 bank 2, Loop 1 mL. Two fractions were collected: one with the product and one with the residue. The relative absorbance Flu₂ (ε₅₀₀ₙₘ=146000 M⁻¹, ε₂₆₀ₙₘ=43350 M⁻¹) and PNA (ε₅₀₀ₙₘ=73000 M⁻¹, ε₂₆₀ₙₘ=104000 M⁻¹) was used to calculate the average conjugation ratio of Flu₂, PNA, and DexVS70. The conjugation ratio of Flu₂ to DexVS70 was 9.4. The conjugation ratio of PNA (sequence AA) to DexVS70 was 1.2.

### Example 4j

### Synthesis of HRP-DexVS70-Seq. AA

Using the procedure of Example 4o below, the conjugate HRP-DexVS70-Seq. AA was made. The ratio of HRP to DexVS70 is 12.2; the ratio of Seq. AA to Dex70 is 1.2.

### Example 4k

### Synthesis of GaM-DexVS70-Seq. AB

The synthesis of GaM-DexVS70-Seq. AB was performed using the procedure in Example 16 with the following changes as indicated.

Dextran (molecular weight 70 kDa) is activated with divinylsulfone to a degree of 92 reactive groups/dextran polymer.

| | |
|---|---|
| 105.0 μL DexVS70 | 7.5 nmol |
| 57.0 μL Goat anti mouse Imunoglobulin (GAM-Ig) | 15 nmol |
| 8.9 μL 4 M NaCl | |
| 10.6 μL 0.8 M NaHCO₃ (pH = 9.5) | |
| 144.5 μL H₂O | |

The above five components were mixed and placed in a water bath at 30° C. for 40 minutes. Two hundred and ninety μL were taken out of the mixture and added to 100 nmol of Seq. AB, which was previously dissolved in 80 μL of H₂O. Then, 20 μL of 0.8 M NaHCO₃ (pH 9.5) was added and the mixture placed in a water bath at 30° C. for 1 hour. Quenching was performed by adding 39 μL of 500 mM cysteine and letting the resultant mixture set for 30 minutes at 30° C.

Purification of the product on FPLC: column SUPERDEX®—200, buffer 10 mM Hepes 100 mM NaCl, method 7 bank 2, Loop 1 mL. Two fractions were collected: one with the product and one with the residue. Relative absorbance PNA(Flu) (ε₅₀₀ₙₘ=73000 M⁻¹) and GAM (ε₂₇₈ₙₘ=213000 M⁻¹) (correction factor for PNA at 278 nm is due to the specific PNA and is calculated: 278/500 nm) was used to calculate the average conjugation ratio of PNA, GAM and DexVS70. The ratio of PNA to DexVS70 was 5.3 and the ratio of GaM to DexVS70 was 0.8.

### Example 4l

### Exemplary Embodiments of PNA1-DexVS-PNA2 Conjugates

TABLE 2

| Conjugate | | | PNA1 | PNA1 to | | PNA2 | PNA2 to | |
|---|---|---|---|---|---|---|---|---|
| designation | ratio | PNA1 | nmol | DexVS | PNA2 | nmol | DexVS | DexVS |
| Conj. CA | 1:9 | Seq. AA | 12.5 | 1.02 | Seq. AD | 100 | 8.2 | DexVS70 |
| Conj. CB | 1:6 | Seq. AC | 40 | 1.5 | Seq. AB | 200 | 7.4 | DexVS70 |
| Conj. CC | 1:16 | Seq. AC | 13.3 | 0.84 | Seq. AB | 200 | 12.7 | DexVS150 |
| Conj. CD | 1:6 | Seq. AC | 40 | 2.3 | Seq. AB | 200 | 11.5 | DexVS150 |

All conjugates were made by standard conjugation procedures below.

### Example 4m

### Synthesis of Anti-Human-BCL2-DexVS70-PNA

Dextran (molecular weight 70 kDa) was activated with divinylsulfone to a degree of 92 reactive groups/dextran polymer, and is designated DexVS70. The antibody Anti-Human-BCL2 is designated AHB.

| | |
|---|---|
| 105 μL DexVS70 | 7.5 nmol |
| 800 μL AHB conc. (2.9 g/L) | 15.1 nmol |
| 25 μL 4 M NaCl | |
| 32 μL 0.8 M NaHCO₃ (pH = 9.5) | |

The above four compounds were mixed and placed in a water bath at 30° C. for 65 minutes. From this mixture, 875 μL was taken out and added to the indicated number of nmol of PNA in the table below; before the addition the PNA had been dissolved in the μL of H₂O indicated in the table below. Then the number of μLs of 0.8 M NaHCO₃ (pH 9.5) was added according to the table below. The resulting mixture was placed in a water bath at 30° C. for 70 minutes. Quenching was performed by adding 6 mg of solid cysteine (0.05 M) to the mixture and letting it stand for 30 minutes at 30° C.

Purification of the product on FPLC: column SUPERDEX®—200, buffer 10 mM Hepes 100 mM NaCl, method 7 bank 2, Loop 1 mL. Two fractions were collected: one with the product and one with the residue. Relative absorbance PNA(Flu) (ε₅₀₀ₙₘ=73000 M⁻¹) and AHB (Pε₂₇₈ₙₘ=213000 M⁻¹) (correction factor for PNA at 278 nm is due to the specific PNA and is calculated: 278/500 nm) was used to calculate the average conjugation ratio of PNA, AHB and DexVS70.

Conjugates with different ratios PNA are shown in the following table.

TABLE 3

| | nmol of | μL of | μL of 0.8 M | | |
|---|---|---|---|---|---|
| Conjugate | PNA | H₂O | NaHCO₃ (pH | PNA to | AHB to |
| designation | added | added | 9.5) added | DexVS70 | DexVS70 |
| Conj. DA | 100 | 75 | 25 | 9.5 | 1.6 |
| Conj. DB | 33 | 30 | 10 | 2.9 | 1.2 |
| Conj. DC | 67 | 60 | 20 | 5.6 | 1.1 |

### Example 4n

### Solid Phase Synthesis and Purification of Lys(Flu)-L₃₀-chr 17:14-L₃₀-Lys(Flu)-L₉₀-Lys(Flu)-L₉₀-Lys(Flu)

All Standard procedures are described below.

1. An MBHA-resin was loaded with Boc-L₃₀-Lys(Fmoc)-L₉₀-Lys(Fmoc)-L₉₀-Lys(Fmoc) using a standard loading procedure to a loading of 0.084 mmol/g.

2. To this resin, Boc-Lys(Fmoc)-L₃₀-AAC-GGG-ATA-ACT-GCA-CCT-was coupled using the peptide synthesizer machine following standard PNA solid phase chemistry. Fmoc protection groups were removed and the amino groups were labeled with fluorescein. After cleaving and precipitation the PNA was dissolved in TFA. The precipitate was washed with ether. The precipitate was dissolved in 200 μL NMP To this solution 6 mg Fmoc-Osu was added and dissolved. Next, DIPEA (9 μL) was added and the reaction was followed using MALDI-TOF mass spectrometry. After 30 minutes the reaction was finished and the PNA was precipitated and washed with ether.

HPLC after dissolving the PNA in 30% CH₃CN and 10% TFA/H₂O gave three pure fractions. The fractions were pooled and lyophilized. The lyophilized PNA was then dissolved in 192 μL NMP. Piperidine (4 μL) and 4 μL DBU was added to this solution which set for 30 minutes. Analysis by MALDI-TOF mass spectrometry gave a molecular weight of 10777.

The precipitate was washed with ether and was then dissolved in 100 μL TFA. The precipitate was washed with ether and then dried using N₂ gas.

### Example 4o

### Standard Synthesis of HRP-DexVS70-PNA Conjugate

Dextran (molecular weight 70 kDa) is activated with divinylsulfone to a degree of 92 reactive groups/dextran polymer.

| | |
|---|---|
| 192 μL DexVS70 | 13.7 nmol |
| 255 μL horse radish peroxidase (HRP) | 602 nmol |
| 15 μL 4 M NaCl | |
| 19 μL 0.8 M NaHCO₃ pH = 9.5 | |
| 119 μL H₂O | |

The above five components are mixed together placed in a water bath at 30° C. for 16 hours. Five hundred microliters of this mixture are added to 50 nmol PNA, which is previously dissolved in 40 μL H₂O. Then, 10 μL of 0.8 M NaHCO₃ (pH 9.5) is added. The mixture is then placed in a water bath at 30° C. for 2 hours. Quenching is performed by adding 55 μL of 110 mM cysteine and letting the resultant mixture set for 30 minutes at 30° C.

Purification of the product is performed by FPLC: column SUPERDEX®—200, buffer 10 mM Hepes 100 mM NaCl, method 7 bank 2, Loop 1 mL.

Two fractions are collected: one with the product and one with the residue. Relative absorbance HRP (ε₄₀₄ₙₘ=83000 M⁻¹, ε₅₀₀ₙₘ=9630 M⁻¹) and PNA(Flu) (ε₅₀₀ₙₘ=73000 M⁻¹) is used to calculate the average conjugation ratio of HRP, PNA and DexVS70.

### Example 4p

### Standard Synthesis of GAM-DexVS70-PNA Conjugate

Dextran (molecular weight 70 kDa) is activated with divinylsulfone to a degree of 92 reactive groups/dextran polymer (DexVS70).

| | |
|---|---|
| 105.0 μL DexVS70 | 7.5 nmol |
| 57.0 μL Goat anti mouse imunoglobulin (GAM) | 15 nmol |
| 8.9 μL 4 M NaCl | |
| 10.6 μL 0.8 M NaHCO₃ (pH = 9.5) | |
| 144.5 μL H₂O | |

The above five components are mixed and placed in a water bath at 30° C. for 40 minutes. Two hundred and ninety μL is taken out of the mixture and added to 50 nmol of PNA, which is previously dissolved in 40 μL of H₂O. Then, 10 μL of 0.8 M NaHCO₃ (pH 9.5) is added and the mixture placed in a water bath at 30° C. for 1 hour. Quenching is performed by adding 34 μL of 500 mM cysteine and letting the resultant mixture set for 30 minutes at 30° C.

Purification of the product on FPLC: column SUPERDEX®—200, buffer 10 mM Hepes 100 mM NaCl, method 7 bank 2, Loop 1 mL. Two fractions are collected: one with the product and one with the residue. Relative absorbance PNA(Flu) (ε₅₀₀ₙₘ=73000 M⁻¹) and GAM (ε₂₇₈ₙₘ=213000 M⁻¹) (correction factor for PNA at 278 nm is due to the specific PNA and is calculated: 278/500 nm) was used to calculate the average conjugation ratio of PNA, GAM and DexVS70.

### Example 4a

### Standard Synthesis of PNA1-DexVS70-PNA2

Dextran (molecular weight 70 kDa) is activated with divinylsulfone to a degree of 92 reactive groups/dextran polymer. PNA1 (100 nmol) is dissolved in 140 μL of DexVS70 (10 nmol). To this mixture 12.5 μL of PNA2 (12.5 nmol) dissolved in H₂O is added, and then 30 μL of NaHCO₃ (pH 9.5) is added and the solution mixed. The resultant mixture is placed in a water bath at 30° C. for 35 minutes. Quenching was performed by adding 18.3 μL of 500 mM cysteine in Hepes and letting this mixture set for 30 minutes at 30° C.

Purification of the product on FPLC: column SUPERDEX®—200, buffer 10 mM Hepes 100 mM NaCl, method 7 bank 2, Loop 1 mL. Two fractions are collected: one with the product and one with the residue. Relative absorbance PNA(Flu) (ε₅₀₀ₙₘ=73000 M⁻¹) and the proportion between the two PNA's is used to calculate the average conjugation ratio of PNA, PNA and DexVS70.

### Example 4r

### Synthesis of the Boc-PNA-I(O-Bz)-Monomer

6-Benzyloxypurine. Sodiumhydride (60% Dispersion in mineral oil; 3.23 g; 80 mmol) was slowly added to benzyl alcohol (30 ml; 34.7 mmol). After the addition of more benzyl alcohol (10 ml) and 6-chloropurine (5.36 g;). The reaction mixture was heated to 100° C. for 4 hours. When the reaction mixture has reached room temperature, water (1 ml) was slowly added. 6-Benzyloxypurine was precipitated by the addition of acetic acid (4.6 ml) and diethylether (550 ml). The precipitate was separated by filtration (11.72 g). Re-crystallization from ether gave (4.78 g; 65.4%). Melting point was 175-177° C. (litt. 170-172° C.)[Ramazaeva N., 1989 #473] 1H-NMR (DMSO-d6): 8.53 (1H, s); 8.39 (1H, s); 7.54-7.35 (5H, m); 5.62 (2H, s).

Methyl (6-(Benzyloxy)purin-9-yl) acetate. 6-Benzyloxypurine (4.18 g; 18.5 mmol) was added to a suspension of potassium carbonate (3.1 g; 22.4 mmol) in DMF (100 ml). After 15 min., bromoacetic acid methyl ester (1.93 ml; 20.4 mmol) was added. The reaction was monitored by TLC in butanol:acetic acid:water 4:1:1. Upon completion, the reaction mixture was partitioned between water (600 ml) and ethyl acetate (600 ml). The organic phase was dried over magnesium sulfate and evaporated to a volume of ˜10 ml and precipitated with pet. ether. The two products were separated by column chromatography using ethyl acetate as the solvent. The products were precipitated in pet. ether. Yield: 2.36 g (43%). Melting point: 111.5-115° C. UV λmax=250 nm (9-alkylated); λmax=260 nm (7-alkylated). 1H-NMR (DMSO-d6):8.60 (1H, s); 8.43 (1H, s); 7.6-7.35 (5H, m); 5.69 (2H, s); 5.26 (2H, s); 3.75 (3H, s).

(6-(Benzyloxy)purin-9-yl) acetic acid. Methyl (6-(Benzyloxy)purin-9-yl) acetate (2.10 g; 7.0 mmol) was dissolved in methanol (70 ml) and 0.1 M NaOH (85 ml) is added. After 15 min. the pH of the reaction mixture was lowered by addition of 0.1 M HCl (˜80 ml) to pH 3. The precipitate was separated from the mixture by filtration and washed with water and ether. Yield: 1.80 g (90.2%). 1H-NMR (DMSO-d6):8.55 (1H, s); 8.37 (1H, s); 7.55-7.30 (5H, m); 5.64 (2H, s); 5.09 (2H, s).

N-((6-(Benzyloxy)purin-9-yl)acetyl)-N-(2-Boc-aminoethyl)glycine. Ethyl N-(2-Boc-aminoethyl)glycinate (0.285 g; 1.15 mmol), (6-(benzyloxy)purin-9-yl) acetic acid (0.284 g; 1.0 mmol) and 3-hydroxy-1,2,3 benzotriazin-4(3H)-one (0.180; 1.1 mmol) was dissolved in dichlormethane/dimethylformamide 1:1 (10 ml). After addition of dicycloehexylcarbodiimide (0.248 g; 1.2 mmol) the reaction was left over night. The precipitate was removed by filtration. The organic phase was extracted twice with saturated sodium bicarbonate, dried with magnesium sulfate and evaporated to a oil. Column purification on silica using dichloromethane with 0-5% methanol as elutant yields the monomer ester which was dissolved in methanol (10 ml). Then, 0.1 M NaOH (12 ml) was added. After 30 min the reaction was filtered and pH adjusted with saturated KHSO4/water (1:3) to 2.7. The water phase was extracted twice with ethyl acetate (2×100 ml). The combined organic phases were dried over magnesium sulfate and evaporated to a volume of 10 ml. Precipitation with pet. ether yielded the monomer (0.15 g; 31%). 1H-NMR (DMSO-d6): 8.51 (1H, s); 8.23 (1H, s); 7.6-7.3 (5H, m); 5.64 (2H, s); 5.31 (ma.)+5.13 (mi.) (2H, s); 4.23 (mi.)+3.98 (ma.) (2H, s); 3.55-3.00 (4H, m); 1.36 (9H, s).

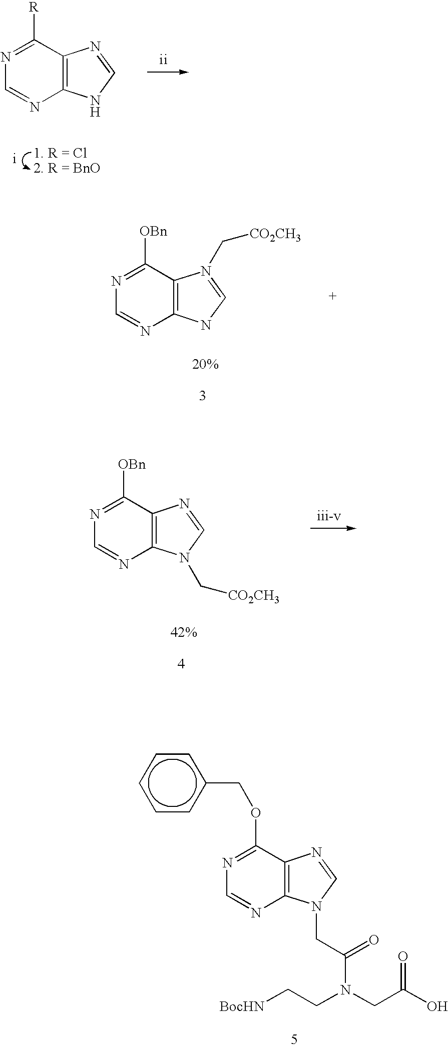

The synthesis of the hypoxanthine PNA monomer. (i) BnOH, NaH (ii) K2CO3, BrCH2CO2CH3 (iii) OH— (iv) DCC, Dhbt-OH, Boc-aeg-OEt (v) OH—

The Boc-PNA-Diaminopurine-(N6-Z)-monomer was prepared according to Gerald Haaima, Henrik F. Hansen, Leif Christensen, Otto Dahl and Peter E. Nielsen; Nucleic Acids Research, 1997, Vol 25, Issue 22 4639-4643.

The Boc-PNA-2-Thiouracil-(S-4-MeOBz)-monomer was prepared according to Jesper Lohse, Otto Dahl and Peter E. Nielsen; Proceedings of the National Academy of Science of the United States of America, 1999, Vol 96, Issue 21, 11804-11808.

The Boc-PNA-Adenine-(Z)-monomer was from PE Biosystems catalog GEN063011.

The Boc-PNA-Cytosine-(Z)-monomer was from PE Biosystems cat. GEN063013.

The Boc-PNA-Guanine-(Z)-monomer was from PE Biosystems cat. GEN063012.

The Boc-PNA-Thymine-monomer was from PE Biosystems cat. GEN063010.

IsoAdenine (2-aminopurine) may be prepared as a PNA-monomer by 9-N alkylation with methylbromoacetate, protection of the amino group with benzylchloroformate, hydrolysis of the methyl ester, carbodiimide mediate coupling to methyl-(2-Boc-aminoethyl)-glycinate, and finally hydrolysis of the methyl ester.

4-thiouracil may be prepared as a PNA-monomer by S-protection with 4-methoxy-benzylchloride, 1-N alkylation with methylbromoacetate, hydrolysis of the methyl ester, carbodiimide mediate coupling to methyl-(2-Boc-aminoethyl)-glycinate, and finally hydrolysis of the methyl ester.

Thiocytosine may be prepared as a PNA monomer by treating the Boc-PNA-cytosine(Z)-monomer methyl ester with Lawessons reagent, followed by hydrolysis of the methyl ester.

A number of halogenated bases are commercially available, and may be converted to PNA monomers analogously to the non-halogenated bases. These include the guanine analog 8-bromo-guanine, the adenine analogs 8-bromo-adenine and 2-fluoro-adenine, the isoadenine analog 2-amino-6-chloro-purine, the 4-thiouracil analog 5-fluoro-4-thio-uracil, and the 2-thiouracil analog 5-chloro-2-thiouracil.

Boc-PNA-Uracil monomers were first described in “Uracil og 5-bromouracil I PNA,” a bachelor project by Kristine Kilså Jensen, Københavns Universitet 1992.

### Example 4s

### Miscellaneous Standard Procedures

a. Loading of resins. P-methyl-BHA-resin (3 g) is loaded with Boc-Lys(Fmoc)-OH 15 mmol/g resin. The lysine is dissolved in NMP and activated with 0.95 equivalents (eq.) HATU and 2 eq. DIPEA. After loading the resin, it is capped by adding a solution of (Ac)₂O/NMP/pyridine (at a ratio of 1/2/2) and letting it set for at least 1 hour or until Kaiser test was negative. After washing with DCM, the resin is dried in a dessicator. Quantitative Kaiser test typically gives a loading of 0.084 mmol/g.

b. Amino Acid Couplings. The Boc protection group is removed from the resin with TFA/m-cresol (at a ratio of 95/5) 2×5 min. The resin is then washed with DCM, pyridine and DMF before coupling with the amino acid, which is dissolved in NMP in a concentration between 0.2 and 0.4 M and activated with 0.95 eq. of HATU and 2 eq of DIPEA for 2 minutes. The coupling is complete when the Kaiser test is negative. Capping occurring by exposing the resin for 3 minutes to (Ac)₂O/pyridine/NMP (at a ratio of 1/2/2). The resin is then washed with DMF and DCM

c. Boc-L₃₀₀-Lys(Fmoc)-resin. To the loaded Boc-Lys(Fmoc)-resin, L₃₀-Linker in a concentration of 0.26 M was coupled using standard amino acid coupling procedure. This was done 10 times giving Boc-L₃₀₀-Lys(Fmoc)-resin.

d. PNA solid phase. On a peptide synthesizer (ABI 433A, Applied Biosystems) PNA monomers are coupled to the resin using standard procedures for amino acid coupling and standard PNA chemistry. Then the resin is handled in a glass vial to remove protections groups and to label with either other amino acids or fluorophores.

Removal of the indicated protection groups is achieved with the following conditions:

Boc: TFA/m-cresol (at a ratio of 95/5) 2×5 min.

Fmoc: 20% piperidine in DMF 2×5 min.

Dde: 3% hydrazine in DMF 2×5 min.

When the synthesis is finished, the PNA is cleaved from the resin with TFA/TFMSA/m-cresol/thioanisol (at a ratio of 6/2/1/1). The PNA is then precipitated with ether and purified on HPLC. MALDI-TOF mass spectrometry is used to determine the molecular weight of the product.

e. Labeling with fluorescein. 5(6)-carboxy fluorescein is dissolved in NMP to a concentration of 0.2 M. Activation is performed with 0.9 eq. HATU and 1 eq. DIPEA for 2 min before coupling for at least 2×20 min or until the Kaiser test is negative.

### Example 4t

### PNA with Positive and Negative Loadings

In order to make better conjugations at one time we tried to give the PNA a loading. Both PNA's were made by PNA standard procedures (See Example 4s above).

1. Flu-L₃₀-Glu-TCA-AGG-TAC-A-Glu-L₃₀₀-Lys(Cys)

Glu=glutamate has negative loadings and for the easiness the PNA is designated −A4−

2. Flu-L₃₀-Lys(Me)₂-TGT-ACC-TTG-A-Lys(Me)₂-L₃₃₀-Lys(cys)

Lys(Me)₂=Boc-Lys(Me)₂-OH has positive loadings and the PNA is designated +T+

TABLE 4

| | | | | | HRP/ | GaM/ | PNA/ |
|---|---|---|---|---|---|---|---|
| name | number | HRP | GaM | equiv. | Dex | Dex | Dex |
| −A4− | D 13041 | D 13050 | | 9 | 12.3 | | 0.13 |
| −A4− | D 13041 | | D 13060 | 7 | | 0.94 | 0.66 |
| +T4+ | D 13042 | D 13058 | | 9 | 13.5 | | 0.19 |
| +T4+ | D 13042 | | D 13056 | 7 | | 1.42 | 0.45 |

As it is shown in the scheme, PNAs with loading are not good at coupling.

### Example 4u

### Target Detection: Procedures Used in the Examples Below

### 1. Fixation of Biological Samples

Tonsil tissue samples were fixed in neutral buffered formalin, NBF (10 mM NaH₂PO₄/Na₂HPO₄, pH 7.0), 145 mM NaCl, and 4% formaldehyde (all obtained from Merck, Whitehouse Station, N.J.). The samples were incubated overnight in a ventilated laboratory hood at room temperature.

### 2. Sample Dehydration and Paraffin Embedding

The tissue samples were placed in a marked plastic histocapsule (Sakura, Japan). Dehydration was performed by sequential incubation in 70% ethanol twice for 45 min, 96% ethanol twice for 45 min, 99% ethanol twice for 45 min, and xylene twice for 45 min. The samples were subsequently transferred to melted paraffin (melting point 56-58° C.) (Merck, Whitehouse Station, N.J.) and incubated overnight (12-16 hours) at 60° C. The paraffin-infiltrated samples were transferred to fresh warm paraffin and incubated for an additional 60 min prior to paraffin embedding in a cast (Sekura, Japan). The samples were cooled to form the final paraffin blocks. The marked paraffin blocks containing the embedded tissue samples were stored at room temperature in the dark.

### 3. Cutting, Mounting and Deparaffination of Embedded Samples

The paraffin blocks were cut and optionally also mounted in a microtome (0355 model RM2065, Feather S35 knives, set at 5.0 micrometer; Leica, Bannockburn, Ill.). The first few millimeters were cut and discarded. Paraffin sections 4-6 micrometers thick were then cut and collected at room temperature. The sections were gently stretched on a 45-60° C. hot water bath before being mounted onto marked microscope glass slides (SUPERFROST® Plus; Fisher, Medford, Mass.), two tissue sections per slide. The slides were then dried and baked in an oven at 60° C. The slides were deparaffinated by incubating twice in xylene for 5 min±2 min twice, then in 96% ethanol for 2 min+/−30 sec, then twice in 70% ethanol for 2 min+/−30 sec, and then once in Tris-buffered saline with TWEEN® (called herein TBST) for 5 min. TBST comprises 50 mM Tris adjusted to pH 7.6 with HCl; 150 mM NaCl; 0.05% TWEEN®20. The slides were deparaffinated by subsequently incubation in xylene twice for 5 min±2 min, 96% ethanol twice for 2 min+/−30 sec and 70% ethanol twice for 2 min+/−30 sec. The slides were immersed in deionized water and left for 1 to 5 min.

### 4. Endogenous Peroxidase Blocking

Samples were incubated with a 3% hydrogen peroxide solution for 5 min. to quench endogenous peroxidase activity, followed by washing in deionized water for 1 to 5 min.

### 5. Antigen Retrieval by Microwave Oven

Antigens in the sample were retrieved by immersing the slides in a container containing Antigen Retrieval Solution, pH 6.0 (DakoCytomation code No. K5204 Vial 7 or optional code No. K5205 Vial 7). The container was closed with a perforated lid and placed in the middle of a microwave oven and left boiling for 10 min. The container was removed from the oven and allowed to cool at room temperature for 20 min. The samples were rinsed in deionized water.

### 6. Antigen Retrieval by Water Bath Incubation

Antigens in the sample were retrieved by immersing the slides in a beaker containing Antigen Retrieval Solution, pH 6.0 (DakoCytomation code No. K5204 Vial 7 or optional code No. K5205 Vial 7). The samples were incubated for 40 min in a water bath at 95-100° C. The beaker was removed from the water bath and allowed to cool at room temperature for 20 min. The samples were rinsed in deionized water.

### 7. Water-Repellent Barrier to Liquids by DakoCytomation Pen

To ensure good coverage of reagent on the tissue sample, the area on the slide with tissue was encircled with a silicone rubber barrier using DakoCytomation Pen (DakoCytomation code No. 2002). The slides were transferred to a rack and placed in a beaker containing Tris-buffered saline with TWEEN® (called herein TBST) and left for 5 min. TBST comprises 50 mM Tris adjusted to pH 7.6 with HCl; 150 mM NaCl; 0.05% TWEEN®20.

### 8. Application of a Primary Antibody

Monoclonal Mouse anti-Human Cytokeratin (DakoCytomation code No. M3515) diluted 1:900 in ChemMate™ Antibody Diluent (DakoCytomation code No. S2022) was applied on the tissue samples and incubated for 30 min in a humid chamber at ambient temperature. The slides were individually rinsed and then washed in TBST for 5 min.

### 9. Application of Three Primary Antibodies

Monoclonal Mouse Anti-Human Cytokeratin (DakoCytomation code No. M3515) diluted 1:300, 1:900 and 1:1600; monoclonal Mouse Anti-Human CD20cy (DakoCytomation code No. M0755) diluted 1:2000, 1:8000 and 1:14000; and monoclonal Mouse Anti-Human Ki-67 Antigen (DakoCytomation code No. M7240) diluted 1:400, 1:1200 and 1:2400 were used. The antibodies were diluted in ChemMate™ Antibody Diluent (DakoCytomation code No. S2022), applied on the tissue samples, and incubated for 30 min in a humid chamber at ambient temperature. The slides were individually rinsed and washed in TBST for 5 min.

### 10. Application of an Antibody/Dextran/PNA1 Conjugate Recognition Unit

Antibody/Dextran/PNA1 conjugate recognition unit is also called “PNA1 conjugate” in the examples that follow. The PNA1 conjugate comprises 70,000 molecular weight dextran. Table 5 summarizes PNA1 conjugates based on a secondary antibody: goat anti-mouse Ig, called herein GAM (DakoCytomation code No. Z0420). Table 6 summarizes PNA1 conjugates based on a primary antibody: mouse anti-human BCL2 oncoprotein, such as Clone 124 (DakoCytomation code No. M0887). The primary antibody was protein A-purified prior to conjugation. The conjugates were diluted in BBA (50 mM Tris adjusted to pH 7.6 with HCl; 150 mM NaCl; 2% BSA; 0.02% bronidox; 2.44 mM 4-aminoantipyrin) and were applied on the tissue sample in a range of dilutions, then incubated for 30 min in a humid chamber at ambient temperature. The slides were individually rinsed and washed in TBST for 5 min.

TABLE 5
PNA1 conjugates useful in indirect recognition
of targets: GAM/Dextran/PNA1

| Conjugate | | | | |
|---|---|---|---|---|
| No. | Sequence | μM Dex | GAM/Dex | PNA1/Dex |
| D14120 | AGA CPT TPG DPT | 1.25 | 1.1 | 4.3 |
| D14102 | GTP TAA TTP PAG | 1.02 | 1.0 | 9.1 |
| D14096 | GTP TAD TTP PAG | 1.15 | 1.4 | 4.2 |
| D14083 | UₛGUₛ DPP TTG D | 0.87 | 0.8 | 5.3 |
| D13171 | UₛGUₛ DPP TTG D | 1.21 | 1.0 | 7.5 |
| D13161 | TTG APP TTA G | 2.11 | 1.1 | 6.0 |
| D13150 | TGT APP TTGA | 2.20 | 1.1 | 4.2 |
| D13102 | TGT ACC TTGA | 2.53 | 1.1 | 2.5 |
| D12102 | TGT ACC TTGA | 2.50 | 1.3 | 4.5 |

TABLE 6
PNA1 conjugates for direct recognition of
targets: anti-BCL2/Dextran/PNA1

| Conjugate | | | | |
|---|---|---|---|---|
| No. | Sequence | μM Dex | Ab/Dex | PNA1/Dex |
| D14128 | UₛGUₛ DPP TTG D | 0.8 | 1.1 | 5.6 |
| D14126 | UₛGUₛ DPP TTG D | 1.0 | 1.2 | 2.9 |
| D14122 | UₛGUₛ DPP TTG D | 1.1 | 1.6 | 9.5 |

In the above tables, the letters A, C, G, U, and T, stand for the natural bases adenine, cytosine, guanine, uracil, and thymine. P stands for pyrimidinone, D for 2,6-diaminopurine, and Uₛ for 2-thiouracil.

11. Fixation of PNA1-Conjugate with 1% Glutardialdehyde

The samples were washed in deionized water for 30 sec. Then, 1% glutardialdehyde (Merck Art. No. 820603), called herein GA, diluted in 22 mM calcium phosphate buffer, pH 7.2, was applied, and the samples were incubated for 10 min in a humid chamber at ambient temperature. The samples were washed in deionized water for 30 sec and in TBST for 5 min.

### 12. Application of a PNA¹-PNA²/Dextran Conjugate Adaptor Unit

PNA¹-PNA²/Dextran conjugate is also called “PNA¹-PNA²” in the following examples. Table 7 summarizes the compositions of PNA¹-PNA² conjugates. PNA¹ is complementary to the PNA1 conjugate, and PNA² is complementary to the PNA2 conjugates D14079 and D13155 described in step 13 below. The sequence of PNA¹ is CUₛGₛGₛDD TUₛD GₛDC and the sequence of PNA² is UₛGUₛ DPP TTG D, in which Uₛ stands for 2-thio-uracil, Gₛ stands for 2-amino-6-thioxopurine, D stands for diaminopurine, and P stands for pyrimidinone. The conjugates, diluted in BBA, were applied to the tissue samples in a range of dilutions, and the samples were then incubated for 30 min in a humid chamber at ambient temperature. The samples were individually rinsed and washed in TBST for 5 min. When testing a PNA¹-PNA² conjugate, fixed concentrations of 0.08 μM PNA1 and 0.05 μM PNA2 were used.

TABLE 7
PNA¹-PNA²/Dextran conjugates

| | Molecular | | | |
|---|---|---|---|---|
| Conjugate | weight | | | |
| No. | of dextran | PNA¹/dex | PNA²/dex | μM PNA¹ |
| D14119 | 150.000 | 2.3 | 11.5 | 4.2 |
| D14106 | 150.000 | 0.8 | 12.7 | 1.3 |
| D14104 | 70.000 | 1.5 | 7.5 | 3.9 |

### 13. Application of Horse Radish Peroxidase/Dextran/PNA2 Conjugate Detection Unit

Horse Radish Peroxidase (HRP)/Dextran/PNA2 conjugates are also called “PNA2 conjugate” in the examples that follow, and are listed in table 8. The PNA2 conjugates comprise 70.000 Da molecular weight dextran. The conjugates diluted in BBA were applied to the tissue samples in a range of dilutions, and samples were incubated for 30 min in a humid chamber at, ambient temperature. The samples were individually rinsed and washed twice in TBST for 5 min.

TABLE 8
PNA2 conjugates: HRP/Dextran/PNA2

| Conjugate | | μM | | |
|---|---|---|---|---|
| No. | Sequence | PNA | HRP/Dex | PNA2/Dex |
| D14133 | TCD DII TAC A | 1.6 | 14.0 | 1.0 |
| D14114 | DGₛT CGₛD DGₛG UₛCUₛ | 3.9 | 11.4 | 2.1 |
| D14110 | DGT CGₛD DGₛG UₛCUₛ | 3.0 | 12.6 | 1.6 |
| D14089 | CUₛGₛ GₛDD TUₛD GₛDC | 2.1 | 14.1 | 1.5 |
| D14086 | UₛCGₛ GₛDD TUₛD GDC | 1.9 | 11.0 | 1.0 |
| D14079 | TCD DGₛGₛ TAC A | 1.9 | 12.2 | 1.2 |
| D13159 | CTA AGₛGₛ TCA A | 1.9 | 12.9 | 1.3 |
| D13155 | TCD DGₛGₛ TAC A | 2.4 | 12.7 | 1.6 |
| D13148 | TCA AGₛGₛ TAC A | 1.9 | 11.6 | 0.8 |
| D13122 | CTA AGG TCA A | 3.2 | 13.0 | 2.1 |
| D13108 | GTG TGT GT | 4.3 | 12.0 | 2.3 |
| D13106 | TCA AGG TAC A | 2.6 | 12.4 | 1.3 |
| D12120 | TCD DGG TAC A | 1.0 | 18.3 | 0.6 |
| D12094 | TCA AGG TAC A | 3.0 | 14.6 | 0.9 |

In table 8, in addition to the nucleobase letter schemes provided for Tables 5-7, I stands for inosine.

### 14. Application of Diaminobenzidine Chromogenic Substrate Solution

The diaminobenzidine chromogenic substrate solution, DAB+ (DakoCytomation code No. K3468) was applied on the tissue samples, and the samples were incubated for 10 min in a humid chamber at ambient temperature. The samples were washed with deionized water for 5 min.

15. Counterstaining with Hematoxylin

The tissue samples were immersed in Mayers Hematoxylin (Bie & Berntsen Code No. LAB00254) for 3 min, rinsed in tap water for 5 min, and finally rinsed with deionized water.

### 16. Cover Slipping

Cover slips were applied to the tissue samples using the aqueous mounting media, Faramount (DakoCytomation code No. S3025).

### 17. Evaluation of the Performance

The tissue staining was examined in a bright field microscope at 10×, 20× or 40× magnification. Both the specific and the non-specific staining intensity were described with a score-system using the range 0 to 3+ with 0.5+ score interval. ChemMate™ EnVision™ Detection kit Rabbit/Mouse (DakoCytomation code No. K5007 bottle A) was used as a reference, and was included in all experiments for testing in parallel with the PNA conjugates. K5007 was used according to manufacturer's instructions. The antibodies were used in the following dilutions: M3515 at 1:900, M0755 at 1:8000, and M7240 at 1:1200. The staining intensity of the K5007 reference using the primary antibody M3515 diluted 1:900 was set to 2+ in order to compare and assess the staining result of the PNA conjugate tested. If the reference deviated more than ±0.5, the test was repeated.

In the examples, the various visualization system combinations of the invention were tested on routine tissue samples. The staining performance was compared with a reference visualization system, using EnVision™ and a very dilute antibody from DakoCytomation. The practical dynamic range of quantitative IHC may be narrow, and e.g. strongly stained (+3) tissues are not easy to compare with respect to intensity. Therefore, on purpose, the staining intensity of the reference system was adjusted to be approximately +2. This was done in order to better monitor and compare differences in staining intensity with the system of the invention.

### Example 4v

### Standard Synthesis of AP-DexVS70-PNA Conjugate

Alkaline Phosphatase (“AP”) (from Calf Intestine, EIA grade) was dialyzed overnight against 2 mM HEPES, pH 7.2; 0.1M NaCl; 0.02 mM. ZnCl2. Dextran (molecular weight 70 kDa) was activated with divinylsulfone to a degree of 92 reactive groups per dextran polymer (DexVS70).

The three components below were mixed together and placed in a water bath at 40° C. for 30 minutes.

| | | |
|---|---|---|
| 192.0 μL DexVS70 | 13.7 | nmol |
| 41.0 μL PNA | 41 | nmol PNA dissolved in H₂O |
| 6.0 μL 1M NaHCO₃ | | |

108.0 μL of the DexVS70-PNA conjugate was taken out and added to a mixture of:

| | |
|---|---|
| 160.0 μL AP | 43.4 nmol |
| 7.7 μL 1M NaHCO₃ | |
| 30.6 μL 20 mM Hepes, pH 7.2; | |
| 1M NaCl; 50 mM MgCl₂; 1 mM ZnCl₂ | |

The mixture was placed in a water bath at 40° C. for 3 hours. Quenching was performed by adding 30.6 μL of 0.1M ethanolamine and letting the mixture stand for 30 minutes in water bath at 40° C. The product was purified on FPLC with: Column Superdex-200, buffer: 2 mM HEPES, pH 7.2; 0.1M NaCl; 5 mM MgCl2; 0.1 mM ZnCl2. Two fractions were collected, one with the product and one with the residue.

In comparison to the experiment described above, another conjugate was made with extended conjugation time. The three components below were mixed together and placed in a water bath at 40° C. for 30 minutes.

| | | |
|---|---|---|
| 192.0 μL DexVS70 | 13.7 | nmol |
| 41.0 μL PNA | 41 | nmol PNA dissolved in H₂O |
| 6.0 μL 1M NaHCO₃ | | |

108.0 μL of the DexVS70-PNA conjugate was taken out and added to a mixture of:

| | |
|---|---|
| 160.0 μL AP | 43.4 nmol |
| 7.7 μL 1M NaHCO₃ | |
| 30.6 μL 20 mM Hepes, pH 7.2; | |
| 1M NaCl; 50 mM MgCl₂; 1 mM ZnCl₂ | |

The mixture was placed in a water bath at 40° C. for 5 hours. Quenching was performed by adding 30.6 μL 0.1M Ethanolamine and letting the mixture stand for 30 minutes in water bath at 40° C. Purification of the product on FPLC: Column Superdex-200, buffer: 2 mM Hepes, pH 7.2; 0.1M NaCl; 5 mM MgCl2; 0.1 mM ZnCl2. Two fractions were collected: One with the product and one with the residue.

Relative absorbance PNA(Flu) (ε500 nm=73000M−1) and AP(ε278 nm=140000M−1. Corrected for absorbance from PNA at 278 nm, this correction factor is due to the specific PNA and it is calculated: 278/500 nm) was used to calculate the average conjugation ratio of PNA, AP and DexVS70.

AP-DexVS70-PNA, 3 hrs:

PNA/DexVS70:1.8

AP/DexVS70:1.8

AP-DexVS70-PNA, 5 hrs:

PNA/DexVS70: 2.0

AP/DexVS70: 2.4

Due to these results, it is recommended to follow a procedure in which the conjugation time (AP+DexVS70-PNA) is 5 hours.

### Example 4w

### Tests of 2 and 3 Layer Visualization Systems

Primary mouse antibody M7240 (Dako) targeting MIB-1 was diluted to final 1:150 in S2022 buffer (Dako) and applied on a multi tissue section. Following 10 minutes incubation at RT the section was washed 5 minutes using 10× diluted S3006 buffer (Dako).

Goat-anti-mouse secondary antibody conjugated with dextran and a first PNA sequence (GaM-dex-PNA1 (218-117)) was diluted to final concentration of 0.08 μM (based on dextran) in BP-HEPES-buffer (1.5% BSA, 3% PEG, 0.15M NaCl, 10 mM HEPES, pH 7.2) and was applied to the section. Following 10 minutes incubation at room temperature (RT), the section was washed 5 minutes using 10× diluted S3006 (Dako). The sections were rinsed in deionized water. Following 10 minutes incubation in 0.5% glutaraldehyde at RT, the sections were rinsed in deionized water and washed 5 minutes using 10× diluted S3006 (Dako).

An adaptor unit comprising dextran coupled to two different PNA sequences, one complementary to PNA1 above (PNA2) and another not complementary to PNA1 (PNA3), called PNA2-dex-PNA3 (218-057) was diluted to a final concentration of 0.05 μM (dextran) in BP-HEPES-buffer (1.5% BSA, 3% PEG, 0.15M NaCl, 10 mM HEPES, pH 7.2) and was applied to the section. Following 10 minutes incubation at RT, the section was washed 5 minutes using 10× diluted S3006 (Dako). Next, a conjugate of a PNA4, complementary to PNA3 above, dextran, and the detectable label alkaline phosphatase (PNA4-dex-AP (209-177)) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2), and was applied. Following minutes incubation at RT, the sections were washed 5 minutes using 10× diluted S3006 (Dako).

Permanent Red working solution (an aqueous Tris buffer with naphthol-phosphate and a diazonium dye; K0640 Dako) was prepared and then applied. Following 10 minutes incubation, the section was washed 5 minutes using 10× diluted S3006 (Dako). Finally the sections were counter stained 5 minutes using haematoxylin S3301 (Dako), rinsed in deionized water, washed 3 minutes in wash buffer, and mounted in Faramount S3025 (Dako). Result: MIB-1=1+/0.5+.

### Example 4x

Primary rabbit antibody A0452 (Dako) targeting CD3 was diluted to final 1:100 in S2022 (Dako) and applied on a multi tissue section. Following 10 minutes incubation at RT the sections were washed 5 minutes using 10× diluted S3006 (Dako).

Then, a goat-anti-rabbit secondary antibody coupled to dextran and a first PNA sequence, PNA2a (GaR-dex-Alexander (209-127)) was diluted to final concentration of 0.08 μM (dextran) in BP-HEPES-buffer (1.5% BSA, 3% PEG, 0.15M NaCl, 10 mM HEPES, pH 7.2) and was applied. Following 10 minutes incubation at RT the sections were washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

Next, a complementary PNA coupled to dextran and detectable label alkaline phosphatase (AP) (PNA2b-dex-AP (209-177) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2) and was applied. Following 10 minutes incubation at RT the section was washed 5 minutes using 10× diluted S3006 (Dako).

Permanent Red working solution (K0640 Dako) was prepared and was applied. Following 10 minutes incubation the sections were washed 5 minutes using 10× diluted S3006 (Dako). Finally the sections were counter stained 5 minutes using haematoxylin S3301 (Dako), rinsed in deionized water, washed 3 minutes in wash buffer, and mounted in Faramount S3025 (Dako). Result: CD3 specific staining=1.5+ compared to non-specific background staining of 1+. The order of detection affects the staining result.

### Example 4v

### Detecting Two Targets in a Sample

General Procedural Note: Before conducting the detection experiment on formalin-fixed, paraffin-embedded (FPPE) tissue sections, the specimen should be deparaffinized (dewaxed), rehydrated, and blocked for endogenous peroxidase activity. Some specimens should be subjected to target retrieval using heat or enzyme digestion. Following target retrieval, the specimens should be rinsed gently with wash buffer.

### Part A. Two-Layer Detection Experiment Using Secondary Antibody Probes

In this experiment, a mouse primary antibody was used as a primary binding agent for a specific target in a tissue sample. That antibody was then recognized by a goat-anti-mouse-dextran-PNA conjugate recognition unit. A different primary antibody, a rabbit antibody, was used as a primary binding agent for a different target in the sample. That antibody was recognized by a goat-anti-rabbit-dextran-PNA recognition unit. One reaction was visualized by a PNA-dextran-HRP (horse-radish peroxidase) detection unit and the other reaction was visualized by a PNA-dextran-AP (alkaline phosphatase) detection unit. PNA sequences 1 and 2 and sequences 3 and 4, respectively, specifically hybridize to each other.

Primary mouse antibody M3515 (Dako) targeting Cytokeratin and primary rabbit antibody Z0311 (Dako) targeting S100 were diluted 1:50 and 1:100 in S2022 buffer (Dako), respectively. The antibodies were applied simultaneously on multi tissue sections. Following 10 minutes incubation at RT the sections were washed 5 minutes using 10× diluted S3006 buffer (Dako). Goat-anti-mouse-dextran-PNA1 (GaM-dex-PNA1) and goat-anti-rabbit-dextran-PNA3 (GaR-dex-PNA3) were both diluted to a final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at room temperature (RT), the sections were washed 5 minutes using 10× diluted S3006 (Dako). The sections were rinsed in deionized water.

The samples were then incubated for 10 minutes in 0.5% glutaraldehyde at RT and then rinsed in deionized water and washed 5 minutes using 10× diluted S3006 (Dako). PNA2-dex-HRP and PNA4-dex-AP were both diluted to final concentration of 0.05 [symbol] M (dextran) in BAP-HEPES-buffer (1.5% BSA, 3% PEG, 0.15M NaCl, 0.05% 4-aminoantipyrin, 10 mM HEPES, pH 7.2). The two conjugates were applied simultaneously on the sections. Following 10 minutes incubation at RT the sections were washed 5 minutes using 10× diluted S3006 (Dako). Permanent Red working solution (K0640 Dako) and DAB+ working solution (an aqueous imidazole buffer with hydrogen peroxide and DAB; K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Result: Cytokeratin=HRP=3+ specific staining and 0 background staining, S100=AP=3+ specific staining and 0 background staining. The order of detection affects the staining result. If detection method 1 is used then Permanent Red dominates. If detection method 2 is used then DAB+ dominates.

### Part B. Two-Layer Detection Experiment Using Antibodies as Probes

Primary mouse antibody M3515 (Dako) targeting Cytokeratin and primary rabbit antibody Z0311 (Dako) targeting S100 were diluted to final 1:50 and 1:400 in S2022 (Dako), respectively. The antibody mixture was applied on multi-tissue sections. Following 10 minutes incubation at RT the sections were washed 5 minutes using 10× diluted S3006 (Dako). GaM-dex-PNA1 (209-149) and GaR-dex-PNA2 (209-127) were both diluted to final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections.

Following 10 minutes incubation at RT the sections were washed 5 minutes using 10× diluted S3006 (Dako). The sections were rinsed in deionized water. Following 10 minutes incubation in 1% glutaraldehyde at RT the sections were rinsed in deionized water and washed 5 minutes using 10× diluted S3006 (Dako). PNA2-dex-HRP (209-157) and PNA4-dex-AP (209-177) were both diluted to final concentration of 0.05 [symbol] M/dex in BAP-HEPES-buffer (1.5% BSA, 3% PEG, 0.15M NaCl, 0.05% 4-aminoantipyrin, 10 mM HEPES, pH 7.2). The two conjugates were applied simultaneously on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Result: Using detection method 1: Cytokeratin=HRP=2.5+ specific staining and 0 background staining, S100=AP=2.5+ specific staining and 0 background staining. Using detection method 2: Cytokeratin=HRP=3+ specific staining and 0.5+ background staining, S100=AP=3+ specific staining and 0 background staining. The order of detection affects the staining result.

### Example 4y

### Further 2 and 3 Layer Systems for Detection of Multiple Targets

### Part A. Combined Two and Three-Layer System

In this example, a mouse antibody primary binding agent was recognized by a GaM-dex-PNA1 and a rabbit antibody primary binding agent was recognized by GaR-dex-PNA2. One reaction was detected by a PNA-dex-Enzyme1 conjugate and the other by a PNA-dex-PNA adaptor unit and then a PNA-dex-Enzyme2 conjugate. PNA1 recognizes PNA2 while PNA3 recognizes PNA4. The enzymes used were HRP and AP, bringing along respectively a brown and red end-product within the same tissue section. The PNA-dex-PNA adaptor unit adds a third layer to the detection system.

Primary mouse antibody M7240 (Dako) targeting MIB-1 and primary rabbit antibody A0452 (Dako) targeting CD3 were diluted to final 1:150 and 1:100 in S2022 (Dako), respectively. The antibody mixture was applied on multi tissue sections. Following 10 minutes incubation at RT the sections were washed 5 minutes using 10× diluted S3006 (Dako).

GaM-dex-PNA1 (218-117) and GaR-dex-PNA3 (209-127) were both diluted to final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at RT the sections were washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA4-dex-HRP (218-021) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako). PNA2-dex-PNA3 (218-057) amplification unit was diluted to final concentration of 0.05 μM (dextran) in BP-HEPES-buffer (1.5% BSA, 3% PEG, 0.15M NaCl, 10 mM HEPES, pH 7.2) and applied on the sections. Following 10 minutes incubation at RT the sections were washed 5 minutes using 10× diluted S3006 (Dako). PNA4-dex-AP (209-177) was diluted to final concentration of 0.05 μM/dex in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Detection method 2: DAB+working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Result: CD3=2-layer experiment=HRP=3+ specific and 0 background staining, MIB-1=3-layer experiment=AP=2+ specific and 1.5+ background staining. The order of detection affects the staining result.

### Part B.

Primary mouse antibody M7240 (Dako) targeting MIB-1 and primary rabbit antibody A0452 (Dako) targeting CD3 were diluted to final 1:150 and 1:100 in S2022 (Dako), respectively. The antibody mixture was applied on multi tissue sections. Following 10 minutes incubation at RT the sections were washed 5 minutes using 10× diluted S3006 (Dako).

GaM-dex-PNA1 (218-117) and GaR-dex-PNA3 (209-127) were both diluted to final concentration of 0.08 [symbol] M (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at RT the sections were washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA4-dex-AP (209-177) was diluted to final concentration of 0.05 [symbol] M (dextran) in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako). PNA2-dex-PNA3 (218-057) was diluted to final concentration of 0.05 [symbol] M (dextran) in BP-HEPES-buffer (1.5% BSA, 3% PEG, 0.15M NaCl, 10 mM HEPES, pH 7.2) and applied on the sections. Following 10 minutes incubation at RT the sections were washed 5 minutes using 10× diluted S3006 (Dako).

PNA4-dex-HRP (218-021) was diluted to final concentration of 0.05 [symbol] M (dextran) in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako).

Permanent Red working solution (K0640 Dako) and DAB+ working solution (K3468 Dako) were prepared. The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water.

Finally the sections were counter stained 5 minutes using haematoxylin S3301 (Dako), rinsed in deionized water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water.

Finally the sections were counter stained 5 minutes using haematoxylin S3301 (Dako), rinsed in deionized water, washed 3 minutes in wash buffer, and mounted in Faramount S3025 (Dako).

Result: Using detection method 1: CD3=2-layer experiment=AP=2+ specific and 0 non-specific, background staining, MIB-1=3-layer experiment=HRP=1.5+ specific and 0 background staining. Using detection method 2: CD3=2-layer experiment=AP=3+ specific and 1.5+ nonspecific staining, MIB-1=3-layer experiment=HRP=1.5+ specific and 1+ background staining. The order of detection affects the staining result.

### Example 4z

### Further Multi-Target Detection Experiment

This example presents a 2-layer detection of two targets in which mouse-Ab-dex-PNA is recognized by PNA-dex-Enzyme1 and rabbit-Ab-dex-PNA is recognized by PNA-dex-Enzyme2. The enzymes are HRP and AP bringing along respectively a brown and red end-product within the same tissue section. As in preceding examples, PNA1 and 2 specifically hybridize, as do PNA3 and 4.

CD3-dex-PNA1 (D16043) and MIB-1-dex-PNA2 (218-097) were both diluted to final concentration of 0.1 [symbol] M (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at RT the sections were washed 5 minutes using 10× diluted S3006 (Dako).

PNA2-dex-HRP (209-141) and PNA4-dex-AP (209-177) were diluted to final concentration of 0.2 M (dextran) and 0.05 [symbol] M (dextran) in BAP-HEPES-buffer (1.5% BSA, 3% PEG, 0.15M NaCl, 0.05% 4-aminoantipyrin, 10 mM HEPES, pH 7.2), respectively. The two conjugates were applied simultaneously on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Result: Using detection method 1: CD3=HRP=2+ specific and 0 non-specific, background staining, MIB-1=AP=3+ specific and 0 background staining. Using detection method 2: CD3=HRP=2+ specific and 0.5+ background staining, MIB-1=AP=2.5+ specific and 0 background staining. The order of detection affects the staining result.

### Example 4aa

### 3-Layer Detection System for Detecting MIB-1 Primary Mouse Antibody

Aim: To show that the MIB-1 primary mouse antibody can be detected in a 3-layer system.

Experimental Steps: Primary mouse antibody M7240 (Dako) targeting MIB-1 was diluted to a final 1:150 in S2022 buffer (Dako) and applied on a multi tissue section. Following 10 minutes incubation at RT the section was washed 5 minutes using 10× diluted S3006 (Dako). GaM-dex-PNA1 was diluted to a final concentration of 0.08 [symbol] M (dextran) in BP-HEPES-buffer (1.5% BSA, 3% PEG, 0.15M NaCl, 10 mM HEPES, pH 7.2) and was applied. Following 10 minutes incubation at RT the section was washed 5 minutes using 10× diluted S3006 buffer (Dako). The sections were rinsed in deionized water.

Following 10 minutes incubation in 0.5% glutaraldehyde at RT the sections were rinsed in deionized water and washed 5 minutes using 10× diluted S3006 (Dako). PNA2-dex-PNA3 was diluted to a final concentration of 0.05 [symbol] M (dextran) in BP-HEPES-buffer (1.5% BSA, 3% PEG, 0.15M NaCl, 10 mM HEPES, pH 7.2) and was applied. (PNA2 hybridizes to PNA1 while PNA3 hybridizes to PNA4.) Following 10 minutes incubation at RT the section was washed 5 minutes using 10× diluted S3006 (Dako).

PNA4-dex-HRP was diluted to final concentration of 0.05 [symbol] M (dextran) in BAP-HEPES-buffer (1.5% BSA, 3% PEG, 0.15M NaCl, 0.05% 4-aminoantipyrin, 10 mM HEPES, pH 7.2) and was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionized water, washed 3 minutes in wash buffer, and mounted in Faramount S3025 (Dako).

Result: The brown end product of the HRP reaction visualize the specific nuclear MIB-1 staining of proliferating cells. Staining intensity score 2.5+ and background score 1+.

### Further Examples of Methods, Systems, and Other Embodiments

### Example 5

A method for automatic selection of a set of reagents to detect one or more targets in a sample, wherein the set of reagents comprises at least two layers for detection of each target, wherein the set comprises at least one reagent that is redundant to another reagent in the set.

The method of Example 5, wherein at least two reagents in the set are redundant to other reagents in the set.

The method of Example 5, wherein the set of reagents detects two or more targets in a sample. The method above, wherein the two or more targets are detected in the same portion of the sample. The method above, wherein the two or more targets are detected separately in different portions of the sample.

The method of Example 5, wherein the detection of the first and/or second target involves three or more layers of detection reagents.

A method for automatic selection of a set of reagents to detect one or more targets in a sample, wherein the set of reagents comprises at least two layers for detection of each target, wherein the set comprises at least one reagent that is redundant to another reagent in the set, and further wherein the set of reagents is selected by a method comprising determining, for each reagent in the set:

the target or targets the reagent may be used to detect;

the layer of the reagent in each target detection method;

the function of the reagent in the detection method;

the other reagents to which the reagent is redundant; and

the other reagents with which the reagent will specifically interact.

The information above could be placed into a computer-generated code, for example, comprising specific information about each detection reagent. Optionally, a computer-generated code may also include other information about the detection reagents such as the incubation conditions, their cross reactivities with other reagents, and information concerning adjustments to the reaction protocol that could be made when using a particular reagent. For instance, the code could note the level of amplification an amplification reagent achieves, whether a blocking agent should be used with the particular detection reagent, and the relative signal strength of a detectable label.

The method of Example 5, which is conducted with the assistance of a computer program. The methods above, wherein a computer-generated code on the containers of the reagents to be selected for the set comprises information used to select the reagents for the set. The methods above, wherein the computer-generated code comprises a bar code.

The methods above, wherein at least one reagent in the set is degenerate. The methods above, wherein the degenerate reagent comprises a degenerate molecular code. The methods above, wherein the degenerate molecular code is a nucleic acid code comprising at least one non-natural nucleic acid base. The methods above, wherein the nucleic acid code is comprised within a segment of PNA or LNA. The methods above, wherein the degenerate molecular code comprises at least one hapten or at least one antigen.

The methods above, wherein the at least two targets are chosen from protein targets, DNA targets, and RNA targets.

### Example 6

A detection apparatus for carrying out any one of the methods above.

### Example 7

A method of detecting one or more targets in a sample, comprising:

(a) obtaining a sample potentially comprising one or more targets;
(b) automatically selecting a set of reagents for detection of the one or more targets,

(i) wherein one of the reagents in the set is redundant to another reagent in the set, and the reagents comprise at least two layers for detection of each target;

(c) contacting the sample with the set of detection reagents;
(d) detecting the presence or absence of signals from the association of the sets of detection reagents with the one or more targets; and
(e) correlating the presence or absence of the signals with the presence or absence of each target in the sample.

The method of Example 7, wherein at least two reagents in the set are redundant to other reagents in the set.

The method of Example 7, wherein the set of reagents detects at least two targets in a sample. The method of Example 7, wherein the set of reagents detects more than two targets in a sample. The methods above, wherein the targets are detected in the same portion of the sample. The methods above, wherein the targets are detected separately in different portions of the sample.

The method of Example 7, wherein the detection of the one or more targets involves three or more layers of detection reagents for at least one target.

The method of Example 7, wherein the set of reagents is selected by a method comprising determining, for each reagent in the set:

the target or targets the reagent may be used to detect;

the layer of the reagent in each target detection method;

the function of the reagent in the detection method;

the other reagents to which the reagent is redundant; and

the other reagents with which the reagent will specifically interact.

The method of Example 7, which is conducted with the assistance of a computer program. The methods above, wherein a computer-generated code on the containers of the reagents to be selected for the set comprises information used to select the reagents for the set.

Optionally, a computer-generated code may also include other information about the detection reagents such as the incubation conditions, their cross reactivities with other reagents, and information concerning adjustments to the reaction protocol that could be made when using a particular reagent. For instance, the code could note the level of amplification an amplification reagent achieves, whether a blocking agent should be used with the particular detection reagent, and the relative signal strength of a detectable label.

The method of Example 7, wherein the computer-generated code comprises a bar code.

The method of Example 7, wherein at least one reagent in the set is degenerate. The method above, wherein the degenerate reagent comprises a degenerate molecular code. A method above, wherein the degenerate molecular code is a nucleic acid code comprising at least one non-natural nucleic acid base. The method above, wherein the nucleic acid code is comprised within a segment of PNA or LNA.

The method of Example 7, wherein the degenerate molecular code comprises at least one hapten or at least one antigen.

The method of Example 7, wherein the targets are chosen from protein targets, DNA targets, and RNA targets.

### Example 8

A detection apparatus for carrying out any one of the methods described above for Example 6 and its variants.

### Example 9

A software algorithm for automated selection of a set of detection reagents according to any one of the methods described in the examples above.

What is claimed is:

1. A system for detecting at least two targets in a sample, the system comprising:
(a) a sample potentially comprising at least two targets;
(b) a set of reagents for detection of the at least two targets, which set is automatically selected
(i) wherein one of the reagents in the set is redundant to another reagent in the set, and the reagents comprise at least two layers for detection of each of the two targets;
(ii) wherein the sample is contacted with the set of detection reagents;
(iii) wherein the presence or absence of signals from the association of the sets of detection reagents with the targets is detected; and
(iv) wherein the presence or absence of the signals is correlated with the presence or absence of targets in the sample.

2. A method comprising automatic selection of a set of reagents to detect one or more targets in a sample, wherein the set of reagents comprises at least two layers for detection of each target, and wherein the set comprises at least one reagent that is redundant to another reagent in the set.

3. The method of claim 2, wherein at least two reagents in the set are redundant to other reagents in the set.

4. The method of claim 2, wherein the set of reagents detects two or more targets in a sample.

5. The method of claim 4, wherein the detection of the first and/or the second target involves three or more layers of detection reagents.

6. The method of claim 2, wherein at the one or more targets are chosen from protein targets, DNA targets, and RNA targets.

7. The method of claim 2, wherein information regarding redundancy of the reagents and information correlating the reagents with the one or more targets is provided in a computer-generated code.

8. The method of claim 7, wherein the computer-generated code comprises a bar code.

9. The method of claim 2, wherein the automatic selection is performed with the assistance of a computer program.

10. The method of claim 2, wherein at least one reagent in the set is degenerate and comprises a degenerate molecular code.

11. The method of claim 10, wherein the degenerate molecular code comprises a nucleic acid code.

12. A method comprising automatic selection of a set of reagents to detect one or more targets in a sample, wherein the set of reagents comprises at least two layers for detection of each target, wherein the set comprises at least one reagent that is redundant to another reagent in the set, and further wherein the automatic selection of the set of reagents comprises determining, for each reagent: the target or targets the reagent may be used to detect; the layer of the reagent in each target detection method; the function of the reagent in the detection method; other reagents in the set to which the reagent is redundant; and other reagents in the set with which the reagent will specifically interact.

13. The method of claim 12, wherein, for each reagent in the set, information regarding: the target or targets the reagent may be used to detect; the layer of the reagent in each target detection method; the function of the reagent in the detection method; other reagents to which the reagent is redundant; and other reagents with which the reagent will specifically interact, is provided in a computer-generated code.

14. The method of claim 13, wherein the computer-generated code comprises a bar code.

15. The method of claim 12, wherein the automatic selection is conducted with the assistance of a computer program.

16. The method of claim 12, wherein the set of reagents detects two or more targets in a sample.

17. The method of claim 16, wherein the detection of the first and/or the second target involves three or more layers of detection reagents.

18. The method of claim 12, wherein at least one reagent in the set is degenerate and comprises a degenerate molecular code.

19. The method of claim 12, wherein the targets are chosen from protein targets, DNA targets, and RNA targets.

20. A method of detecting one or more targets in a sample, comprising:
(a) obtaining a sample potentially comprising one or more targets;
(b) automatically selecting a set of reagents for detection of the one or more targets,
(i) wherein one of the reagents in the set is redundant to another reagent in the set, and the reagents comprise at least two layers for detection of each target;
(c) contacting the sample with the set of detection reagents;
(d) detecting the presence or absence of signals from the association of the sets of detection reagents with the one or more targets; and
(e) correlating the presence or absence of the signals with the presence or absence of each target in the sample.

21. The method of claim 20, wherein at least two reagents in the set are redundant to other reagents in the set.

22. The method of 20, wherein the set of reagents detects at least two targets in a sample.

23. The method of claim 20, wherein the detection of the one or more targets involves three or more layers of detection reagents for at least one target.

24. The method of 20, wherein the set of reagents is selected by a method comprising determining, for each reagent in the set:
the target or targets the reagent may be used to detect; the layer of the reagent in each target detection method; the function of the reagent in the detection method; other reagents to which the reagent is redundant; and other reagents with which the reagent will specifically interact.

25. The method of claim 20, wherein the automatic selection is conducted with the assistance of a computer program.

26. The method of claim 25, wherein the computer-generated code comprises a bar code.

27. The method of claim 20, wherein at least one reagent in the set is degenerate and comprises a degenerate molecular code.

28. The method of claim 27, wherein the degenerate molecular code is a nucleic acid code.

29. The method of claim 20, wherein the one or more targets are chosen from protein targets, DNA targets, and RNA targets.

30. A detection apparatus for carrying out the method according to claim 2.

31. The apparatus of claim 30, wherein the function of the apparatus is controlled at least in part by a computer algorithm.

32. A software algorithm for automated selection of a set of detection reagents according the method of claim 2.

33. A detection apparatus for carrying out the method according to claim 12.

34. A detection apparatus for carrying out the method according to claim 20.

35. A software algorithm for automated selection of a set of detection reagents according to claim 12.

36. A software algorithm for automated selection of a set of detection reagents according to claim 20.
